(19) United States
(12) Patent Application Publication
(10) Pub. No.: US 2025/0215180 A1
(43) Pub. Date: Jul. 3, 2025

(54) HEAT INTEGRATION IN A PROCESS FOR HYDROLYTICALLY DEPOLYMERIZING A POLYAMIDE

(71) Applicant: BASF SE, Ludwigshafen am Rhein (DE)

(72) Inventors: Stefan BLEI, Ludwigshafen am Rhein (DE); Faissal-Ali EL-TOUFAILI, Ludwigshafen am Rhein (DE); Zeljko KOTANJAC, Ludwigshafen am Rhein (DE); Vikram Raghavendhar RAVIKUMAR, Ludwigshafen am Rhein (DE)

(21) Appl. No.: 18/852,582

(22) PCT Filed: Mar. 30, 2023
(86) PCT No.: PCT/EP2023/058260

§ 371 Date: Sep. 30, 2024

(30) Foreign Application Priority Data
Apr. 1, 2022 (EP) 22166311.5

(51) Int. Cl. C08J 11/14

(52) U.S. Cl. CPC C08J 11/14; C08J 2377/02

(57) ABSTRACT

The present invention relates to a heat-integrated process for hydrolytically depolymerizing a polyamide prepared from ε-caprolactam, said polyamide being contained in a solid material M.

The present invention relates to a heat-integrated process for hydrolytically depolymerizing a polyamide prepared from ε-caprolactam.

Polyamide, and in particular polyamide 6 being characterized by the formula (—NH—(CH₂)₅—CO—)ₙ, can be found in numerous materials, such as packaging, engineering plastics from automotive and textile filaments. The latter represents about 40% of the polyamide 6 global market. At present, only a very small part of the textile filaments is recycled while it represents a significant percentage of the global CO₂ emissions. There is thus a need to recycle polyamide 6 from such materials. Processes for alkaline depolymerizing a polyamide exists. Further, the processes in the art are energy-intensive processes. Thus, there is a need to provide an improved process for depolymerizing a polyamide able to overcome these issues.

Surprisingly, it was found that the process of the present invention according to which polyamide is hydrolytically depolymerized is more robust and is more cost effective. Further, the process of the present invention permits to reduce the overall energy consumption compared to known processes for depolymerization of a polyamide which also permits to reduce costs. Hence, using a heat-integrated process for depolymerizing a polyamide according to the present invention permits to reduce the CO₂ footprint.

Therefore, the present invention relates to a heat-integrated process for hydrolytically depolymerizing a polyamide prepared from ε-caprolactam, said polyamide being contained in a solid material M, the process comprising
(i) preparing an aqueous liquid stream S_{R} comprising ε-caprolactam dissolved in water, comprising
   (i.1) providing the solid material M comprising the polyamide, and providing a aqueous liquid stream S_{W};
   (i.2) preparing a mixture of the solid material M and the aqueous liquid stream S_{W} provided according to (i.1);
   (i.3) preparing an aqueous mixture M_{A} comprising the polyamide dissolved in water from the mixture prepared according to (i.2);
   (i.4) subjecting the aqueous mixture M_{A} prepared according to (i.3) to depolymerization conditions comprising a depolymerization temperature T_{D} at a depolymerization pressure p_{D} in a chemical reactor unit R_{U}, obtaining a liquid aqueous reaction mixture M_{R} in R_{U}, M_{R} comprising ε-caprolactam dissolved in water, wherein T_{D} is in the range of from 230 to 320° C. and p_{D} is in the range of from 40 to 120 bar;
   (i.5) removing an aqueous liquid stream S_{R} from R_{U}, S_{R} comprising ε-caprolactam dissolved in water, wherein said aqueous liquid stream S_{R} has a pressure p_{R} in the range of from 40 to 120 bar and a temperature T_{R} in the range of from 230 to 320° C.;
   (i.6) optionally passing the aqueous liquid stream S_{R} through a heat exchanging unit HU_{O}, obtaining an aqueous liquid stream S_{LO} having a temperature T_{LO} with T_{LO}<T_{R} and having a pressure p_{LO} in the range of from 40 to 120 bar;
(ii) subjecting the aqueous liquid stream S_{R} obtained according to (i.5), or the aqueous liquid stream S_{LO} obtained according to (i.6), to depressurization in an evaporation unit EU, obtaining from EU at least one aqueous vapor stream S_{V} having a temperature T_{V} in the range of from 100 to 230° C., and at least one aqueous liquid stream S_{L} comprising ε-caprolactam dissolved in water;
(iii) passing at least one aqueous vapor stream S_{V} obtained according to (ii), having a temperature T_{V}, through a heat exchanging unit HU, thereby heating in HU at least one stream S_{T} having a temperature T_{ST}, obtaining a cooled aqueous stream S_{V}, S_{CV}, having a temperature T_{CV} with T_{CV}<T_{V} and at least one heated stream S_{T}, S_{HT}, having a temperature T_{HST} with T_{HST}>T_{ST};
(iv) separating ε-caprolactam from at least one aqueous liquid stream S_{L} obtained according to (ii) in at least one heat consuming purification unit PU, wherein the heat consumed in PU is at least partially provided by at least one heated stream S_{HT};
(v) recycling at least one aqueous liquid stream S_{RE} obtained from at least one heat consuming purification unit PU according to (iv) into R_{U}, preferably as part of the aqueous liquid stream S_{W} according to (i.1).

Preferably, the mixture prepared according to (i.2) is prepared in the chemical reactor unit R_{U}.

Preferably, according to (i.5), the aqueous liquid stream S_{R} has a pressure p_{R} in the range of from 50 to 100 bar, more preferably in the range of from 60 to 90 bar.

Preferably, according to (i.5), the aqueous liquid stream S_{R} has a temperature T_{R} in the range of from 250 to 310° C., more preferably in the range of from 270 to 300° C.

Preferably, the solid material M provided according to (i.1) comprises, more preferably consists of, waste material, wherein said waste material more preferably comprises textile waste material.

Preferably, the process comprises
(ii) subjecting the aqueous liquid stream S_{R} obtained according to (i.5), or the aqueous liquid stream S_{LO} obtained according to (i.6), to depressurization in an evaporation unit EU comprising n evaporation sub-units EU(j), 1≤j≤n with n≥1, obtaining from each sub-unit EU(j) an aqueous vapor stream S_{V}(j) and an aqueous liquid stream S_{L}(j) comprising ε-caprolactam dissolved in water, wherein S_{V}(j) has a temperature T_{V}(j) and a pressure p_{V}(j) and wherein S_{L}(j) has a temperature T_{L}(j) and a pressure p_{L}(j);
(iii) passing at least one aqueous vapor stream S_{V}(j) obtained according to (ii) and having a temperature T_{V}(j) in the range of from 100 to 230° C., through a heat exchanging unit HU(j), thereby heating in HU(j) a stream S_{T}(j) having a temperature T_{ST}(j), obtaining a cooled aqueous stream S_{V}(j), S_{CV}(j), having a temperature T_{CV}(j) with T_{CV}(j)<T_{V}(j) and at least one heated stream S_{T}(j), S_{HT}(j), having a temperature T_{HST}(j) with T_{HST}(j)>T_{ST}(j);
(iv) separating ε-caprolactam from at least one aqueous liquid stream S_{L}(j) obtained according to (ii) in at least one heat consuming purification unit PU, wherein the heat consumed in PU is at least partially provided by at least one heated stream S_{HT}(j);
(v) recycling at least one aqueous liquid stream S_{RE} obtained from at least one heat consuming purification unit PU according to (iv) into R_{U}, more preferably as part of the aqueous liquid stream S_{W} according to (i.1);
wherein at least one evaporation sub-unit EU(j), more preferably every evaporation sub-unit EU(j) according to (ii) comprises, more preferably consists of, a flash drum.

Preferably, according to (ii), the aqueous liquid stream S_{R} obtained according to (i.5), or the aqueous liquid stream S_{LO} obtained according to (i.6), is subjected to depressurization in the evaporation unit EU(1); and, according to (iv), ε-caprolactam is separated from the aqueous liquid stream S_{L}(n) obtained according to (ii) in at least one heat consuming purification unit PU.

Preferably, n>1, and wherein for j<n, the process comprises passing the aqueous liquid stream S_{L}(j) obtained from the evaporation sub-unit EU(j) as feed stream into the evaporation sub-unit EU(j+1). More preferably, the n evaporation sub-units EU(j) are serially coupled.

Preferably, n>1, and wherein for j<n, T_{V}(j+1)<T_{V}(j); p_{V}(j+1)<p_{V}(j); T_{L}(j+1)<T_{L}(j); and p_{L}(j+1)<p_{L}(j).

Preferably, p_{V}(n) is in the range of from 0.90 to 1.5 bar, more preferably in the range of from 0.95 to 1.1 bar; T_{V}(n) is in the range of from 90 to 110° C., more preferably in the range of from 95 to 100° C.; p_{L}(n) is in the range of from 0.90 to 1.5 bar, more preferably in the range of from 0.95 to 1.1 bar; and T_{L}(n) is in the range of from 90 to 110° C., more preferably in the range of from 95 to 105° C.

### Heat Exchange Upstream of EU According to (i.6)

Preferably, n is in the range of from 1 to 3, more preferably n=1 or n=2, more preferably n=1.

The process preferably comprises passing the aqueous liquid stream S_{R} through a heat exchanging unit HU_{O} according to (i.6), obtaining an aqueous liquid stream S_{LO} having a temperature T_{LO} with T_{LO}<T_{R} and having a pressure p_{LO} in the range of from 40 to 120 bar; and subjecting the aqueous liquid stream S_{LO} obtained according to (i.6) to depressurization according to (ii).

Preferably, T_{LO} is in the range of from 130 to 150° C., more preferably in the range of from 120 to 140° C. and a pressure pro is in the range of from 40 to 120 bar, more preferably in the range of from 50 100 bar, more preferably in the range of from 60 to 90 bar.

Preferably, the aqueous vapor stream S_{V}(n) obtained from EU(n) has a pressure p_{V}(n) in the range of from 0.95 to 1.5 bar, more preferably in the range of from 1.0 to 1.4 bar, and a temperature T_{V}(n) in the range of from 90 to 110° C., more preferably in the range of from 95 to 100° C.; and the aqueous liquid stream S_{L}(n) obtained from EU(n) has a pressure p_{L}(n) in the range of from 0.95 to 1.5 bar, more preferably in the range of from 1.0 to 1.4 bar, and a temperature T_{L}(n) in the range of from 90 to 110° C., more preferably in the range of from 95 to 100° C. Preferably, for n=1 according to a preferred aspect, the aqueous vapor stream S_{V}(1) obtained from EU(1) has a pressure p_{V}(1) in the range of from 0.95 to 1.5 bar, more preferably in the range of from 1.0 to 1.4 bar, and a temperature T_{V}(1) in the range of from 90 to 110° C., more preferably in the range of from 95 to 100° C.; and the aqueous liquid stream S_{L}(1) obtained from EU(1) has a pressure p_{L}(1) in the range of from 0.95 to 1.5 bar, more preferably in the range of from 1.0 to 1.4 bar, and a temperature T_{L}(1) in the range of from 90 to 110° C., more preferably in the range of from 95 to 100° C.

Preferably, according to (iv), ε-caprolactam is separated from the aqueous liquid stream S_{L}(1) obtained according to (ii) in at least one heat consuming purification unit PU, wherein the heat consumed in PU is at least partially provided by the heated stream S_{HT}(1).

Preferably, the process further comprises recycling the cooled aqueous stream S_{CV}(1) into R_{U}, more preferably as part of the aqueous liquid stream S_{W} according to (i.1).

The presence of a heat exchanger upstream of the evaporation unit EU according to an alternative of the present invention is shown in FIG. 1.

### No Heat Exchange Upstream of EU(1)

Alternatively, the aqueous liquid stream S_{R} is preferably not passed through a heat exchanging unit HU_{O} according to (i.6), and, according to (ii), the aqueous liquid stream S_{R} obtained according to (i.5) is subjected to depressurization in an evaporation unit EU.

Preferably, n is in the range of from 2 to 5, more preferably in the range of from 2 to 4, more preferably n=2 or n=3.

Preferably, the process comprises
(ii) subjecting the aqueous liquid stream S_{R} obtained according to (i.5) to depressurization in an evaporation unit EU comprising n evaporation sub-units EU(j), obtaining from the sub-unit EU(j) an aqueous vapor stream S_{V}(j) and an aqueous liquid stream S_{L}(j) comprising ε-caprolactam dissolved in water, wherein S_{V}(j) has a temperature T_{V}(j) and a pressure p_{V}(j) and wherein S_{L}(j) has a temperature T_{L}(j) and a pressure p_{L}(j);
   wherein for j<n, the aqueous liquid stream S_{L}(j) obtained from the evaporation sub-unit EU(j) is passed as feed stream into the evaporation sub-unit EU(j+1);
   wherein for j<n, T_{V}(j+1)<T_{V}(j); p_{V}(j+1)<p_{V}(j); T_{L}(j+1)<T_{L}(j); and p_{L}(j+1)<p_{L}(j);
   wherein p_{V}(n) is in the range of from 0.95 to 1.5 bar, more preferably in the range of from 1.0 to 1.4 bar; T_{V}(n) is in the range of from 90 to 110° C., more preferably in the range of from 95 to 100° C.; p_{L}(n) is in the range of from 0.95 to 1.5 bar, more preferably in the range of from 1.0 to 1.4 bar; and T_{L}(n) is in the range of from 90 to 110° C., more preferably in the range of from 95 to 105° C.;
(iii) passing at least the aqueous vapor stream S_{V}(n) obtained according to (ii) through the heat exchanging unit HU(n), thereby heating in HU(n) the stream S_{T}(n) having a temperature T_{ST}(n), obtaining the cooled aqueous stream S_{V}(n), S_{CV}(n), having a temperature T_{CV}(n) with T_{CV}(n)<T_{V}(n) and the heated stream S_{T}(n), S_{HT}(n), having a temperature T_{HST}(n) with T_{HST}(n)>T_{ST}(n);
(iv) separating ε-caprolactam from the aqueous liquid stream S_{L}(n) obtained according to (ii) in at least one heat consuming purification unit PU, wherein the heat consumed in PU is at least partially provided by at least one of the heated streams S_{HT}(j);
(v) recycling at least one aqueous liquid stream S_{RE} obtained from at least one heat consuming purification unit PU according to (iv) into R_{U}, more preferably as part of the aqueous liquid stream S_{W} according to (i.1).

Preferably, according to (iii), at least the aqueous vapor streams S_{V}(j) for j=2 . . . n obtained according to (ii) are passed through the heat exchanging units HU(j), j=2 . . . n.

The absence of a heat exchanger upstream of the evaporation unit EU according to an alternative of the present invention is shown in FIGS. 2-5. Said alternative is further detailed in the following.

According to a first preferred aspect, the process preferably comprises
(ii) subjecting the aqueous liquid stream S_{R} obtained according to (i.5) to depressurization in an evaporation unit EU comprising n evaporation sub-units EU(j), obtaining from the sub-unit EU(j) an aqueous vapor stream S_{V}(j) and an aqueous liquid stream S_{L}(j) comprising ε-caprolactam dissolved in water, wherein S_{V}(j) has a temperature T_{V}(j) and a pressure p_{V}(j) and wherein S_{L}(j) has a temperature T_{L}(j) and a pressure p_{L}(j);
   wherein the aqueous liquid stream S_{R} is passed as feed stream into the evaporation sub-unit EU(1);
   wherein for j<n, the aqueous liquid stream S_{L}(j) obtained from the evaporation sub-unit EU(j) is passed as feed stream into the evaporation sub-unit EU(j+1);
   wherein for j<n, T_{V}(j+1)<T_{V}(j); p_{V}(j+1)<p_{V}(j); T_{L}(j+1)<T_{L}(j); and p_{L}(j+1)<p_{L}(j);
   wherein p_{V}(n) is in the range of from 0.95 to 1.5 bar, more preferably in the range of from 1.0 to 1.4 bar; T_{V}(n) is in the range of from 90 to 110° C., more preferably in the range of from 95 to 100° C.; p_{L}(n) is in the range of from 0.95 to 1.5 bar, more preferably in the range of from 0.95 to 1.4 bar; and T_{L}(n) is in the range of from 90 to 110° C., more preferably in the range of from 95 to 105° C.;
(iii) passing the aqueous vapor streams S_{V}(j) obtained according to (ii) through the heat exchanging units HU(j), thereby heating in HU(j) the stream S_{T}(j) having a temperature T_{ST}(j), obtaining the cooled aqueous stream S_{V}(j), S_{CV}(j), having a temperature T_{CV}(j) with T_{CV}(j)<T_{V}(j) and the heated stream S_{T}(j), S_{HT}(j), having a temperature T_{HST}(j) with T_{HST}(j)>T_{ST}(j);
(iv) separating ε-caprolactam from the aqueous liquid stream S_{L}(n) obtained according to (ii) in at least one heat consuming purification unit PU, wherein the heat consumed in PU is at least partially provided by at least one of the heated streams S_{HT}(j);
(v) recycling at least one aqueous liquid stream S_{RE} obtained from at least one heat consuming purification unit PU according to (iv) into R_{U}, more preferably as part of the aqueous liquid stream S_{W} according to (i.1).

Preferably, according to said first aspect, n=2 and
p_{V}(1) is in the range of from 7.5 to 18 bar, more preferably in the range of from 8 to 16 bar;
T_{V}(1) is in the range of from 160 to 210° C., more preferably in the range of from 170 to 200° C.;
p_{L}(1) is in the range of from 7.5 to 18 bar, more preferably in the range of from 8 to 16 bar; and
T_{L}(1) is in the range of from 160 to 210° C., more preferably in the range of from 170 to 200° C.

Alternatively, preferably, n=3 and
p_{V}(1) is in the range of from 19 to 31 bar, more preferably in the range of from 20 to 30 bar;
T_{V}(1) is in the range of from 205 to 240° C., more preferably in the range of from 210 to 235° C.;
p_{L}(1) is in the range of from 19 to 31 bar, more preferably in the range of from 20 to 30 bar; and
T_{L}(1) is in the range of from 205 to 240° C., more preferably in the range of from 210 to 135° C.;
p_{V}(2) is in the range of from 7.5 to 18 bar, more preferably in the range of from 8 to 16 bar;
T_{V}(2) is in the range of from 160 to 210° C., more preferably in the range of from 170 to 200° C.;
p_{L}(2) is in the range of from 7.5 to 18 bar, more preferably in the range of from 8 to 16 bar; and
T_{L}(2) is in the range of from 160 to 210° C., more preferably in the range of from 170 to 200° C.; or
p_{V}(1) is in the range of from 7.5 to 18 bar, more preferably in the range of from 8 to 16 bar;
T_{V}(1) is in the range of from 160 to 210° C., more preferably in the range of from 170 to 200° C.;
p_{L}(1) is in the range of from 7.5 to 18 bar, more preferably in the range of from 8 to 16 bar; and
T_{L}(1) is in the range of from 160 to 210° C., more preferably in the range of from 170 to 200° C.;
p_{V}(2) is in the range of from 1.5 to 7 bar, more preferably in the range of from 2 to 7 bar;
T_{V}(2) is in the range of from 115 to 165° C., more preferably in the range of from 120 to 165° C.;
p_{L}(2) is in the range of from 1.5 to 7 bar, more preferably in the range of from 2 to 7 bar; and T_{L}(2) is in the range of from 115 to 165° C., more preferably in the range of from 120 to 165° C.

According to said first preferred aspect, the process preferably further comprises recycling at least one of the cooled aqueous streams S_{CV}(j), more preferably all cooled aqueous streams S_{CV}(j) obtained according to (iii) into R_{U}, more preferably as part of the aqueous liquid stream S_{W} according to (i.1).

Said first aspect of the present invention is shown in FIG. 2.

According to a second preferred aspect, the process preferably comprises
(ii) subjecting the aqueous liquid stream S_{R} obtained according to (i.5) to depressurization in an evaporation unit EU comprising n evaporation sub-units EU(j), obtaining from the sub-unit EU(j) an aqueous vapor stream S_{V}(j) and an aqueous liquid stream S_{L}(j) comprising ε-caprolactam dissolved in water, wherein S_{V}(j) has a temperature T_{V}(j) and a pressure p_{V}(j) and wherein S_{L}(j) has a temperature T_{L}(j) and a pressure p_{L}(j);
   wherein the aqueous liquid stream S_{R} is passed as feed stream into the evaporation sub-unit EU(1);
   wherein for j<n, the aqueous liquid stream S_{L}(j) obtained from the evaporation sub-unit EU(j) is passed as feed stream into the evaporation sub-unit EU(j+1);
   wherein for j<n, T_{V}(j+1)<T_{V}(j); p_{V}(j+1)<p_{V}(j); T_{L}(j+1)<T_{L}(j); and p_{L}(j+1)<p_{L}(j);
   wherein p_{V}(n) is in the range of from 0.95 to 1.5 bar, more preferably in the range of from 1.0 to 1.4 bar; T_{V}(n) is in the range of from 90 to 110° C., more preferably in the range of from 95 to 100° C.; p_{L}(n) is in the range of from 0.95 to 1.5 bar, more preferably in the range of from 1.0 to 1.4 bar; and T_{L}(n) is in the range of from 90 to 110° C., more preferably in the range of from 95 to 105° C.;
(iii) passing the aqueous vapor streams S_{V}(j), j>1, obtained according to (ii) through the heat exchanging units HU(j), thereby heating in HU(j) the stream S_{T}(j) having a temperature T_{ST}(j), obtaining the cooled aqueous stream S_{V}(j), S_{CV}(j), having a temperature T_{CV}(j) with T_{CV}(j)<T_{V}(j) and the heated stream S_{T}(j), S_{HT}(j), having a temperature T_{HST}(j) with T_{HST}(j)>T_{ST}(j);
(iv) separating ε-caprolactam from the aqueous liquid stream S_{L}(n) obtained according to (ii) in at least one heat consuming purification unit PU, wherein the heat consumed in PU is at least partially provided by at least one of the heated streams S_{HT}(j);
(v) recycling at least one aqueous liquid stream S_{RE} obtained from at least one heat consuming purification unit PU according to (iv) into R_{U}, more preferably as part of the aqueous liquid stream S_{W} according to (i.1);
   wherein x % of the stream S_{V}(1) obtained according to (ii) are admixed with at least one of the streams S_{RE} according to (v), 0<x≤100;
   wherein, if x≠100, the process comprises dividing the stream S_{V}(1) into a substream S_{V1}(1) and a substream S_{V2}(1), S_{V1}(1) constituting x % of the stream S_{V}(1) and S_{V2}(1) constituting (100-x) % of the stream S_{V}(1), and admixing S_{V1}(1) with at least one of the streams S_{RE} according to (v).

Preferably, for x≠100, the substream S_{V2}(1) is passed through the heat exchanging unit HU(1), thereby heating in HU(1) the stream S_{T}(1) having a temperature T_{ST}(1), obtaining the cooled aqueous stream S_{V1}(1), S_{CV}(1), having a temperature T_{CV}(1) with T_{CV}(1)<T_{V1}(1) and the heated stream S_{T}(1), S_{HT}(1), having a temperature T_{HST}(1) with T_{HST}(1)>T_{ST}(1).

Said second aspect is shown in FIGS. 3 and 5. Said aspect is further detailed in the following.

As a first alternative of the second aspect of the present invention, preferably 25≤x≤100, more preferably 50≤x≤100, more preferably 75≤x≤100, wherein more preferably, x=100.

Preferably, according to said first alternative of the second aspect, n=3 and
p_{V}(1) is in the range of from 19 to 31 bar, more preferably in the range of from 20 to 30 bar;
T_{V}(1) is in the range of from 205 to 240° C., more preferably in the range of from 210 to 235° C.;
p_{L}(1) is in the range of from 19 to 31 bar, more preferably in the range of from 20 to 30 bar; and
T_{L}(1) is in the range of from 205 to 240° C., more preferably in the range of from 210 to 135° C.;
p_{V}(2) is in the range of from 7.5 to 18 bar, more preferably in the range of from 8 to 16 bar;
T_{V}(2) is in the range of from 160 to 210° C., more preferably in the range of from 170 to 200° C.;
p_{L}(2) is in the range of from 7.5 to 18 bar, more preferably in the range of from 8 to 16 bar; and
T_{L}(2) is in the range of from 160 to 210° C., more preferably in the range of from 170 to 200° C.; or
p_{V}(1) is in the range of from 7.5 to 18 bar, more preferably in the range of from 8 to 16 bar;
T_{V}(1) is in the range of from 160 to 210° C., more preferably in the range of from 170 to 200° C.;
p_{L}(1) is in the range of from 7.5 to 18 bar, more preferably in the range of from 8 to 16 bar; and
T_{L}(1) is in the range of from 160 to 210° C., more preferably in the range of from 170 to 200° C.;
p_{V}(2) is in the range of from 1.5 to 7 bar, more preferably in the range of from 2 to 7 bar;
T_{V}(2) is in the range of from 115 to 165° C., more preferably in the range of from 120 to 165° C.;
p_{L}(2) is in the range of from 1.5 to 7 bar, more preferably in the range of from 2 to 7 bar; and T_{L}(2) is in the range of from 115 to 165° C., more preferably in the range of from 120 to 165° C.

According to said first alternative, preferably, admixing x % of the stream S_{V}(1) obtained according to (ii) with at least one of the streams S_{RE} according to (v) comprises bubbling x % of the stream S_{V}(1) into the at least one aqueous liquid stream S_{RE}.

According to said first alternative, the process preferably further comprises recycling at least one of the cooled aqueous streams S_{CV}(j), more preferably all cooled aqueous streams S_{CV}(j) obtained according to (iii) into R_{U}, more preferably as part of the aqueous liquid stream S_{W} according to (i.1).

Said first alternative of the second aspect of the present invention is shown in FIG. 3.

According to a second alternative of the second aspect of the present invention, preferably, 1≤x≤20, more preferably 2≤x≤15, more preferably 5≤x≤10.

According to said second alternative, preferably, n=2 and
p_{V}(1) is in the range of from 7.5 to 18 bar, more preferably in the range of from 8 to 16 bar;
T_{V}(1) is in the range of from 160 to 210° C., more preferably in the range of from 170 to 200° C.;
p_{L}(1) is in the range of from 7.5 to 18 bar, more preferably in the range of from 8 to 16 bar; and
T_{L}(1) is in the range of from 160 to 210° C., more preferably in the range of from 170 to 200° C.

Preferably, according to said second alternative, admixing x % of the stream S_{V}(1) obtained according to (ii) with at least one of the streams S_{RE} according to (v) comprises passing the substream S_{V1}(1) through a heat exchanging unit HU_{RE}, thereby cooling the substream S_{V1}(1), wherein in the heat exchanging unit HU_{RE}, at least one of the streams S_{RE} is heated prior to being recycled into R_{U} according to (v).

Preferably, according to said second alternative, when x=100, the process comprises passing the substream S_{V2}(1) through a heat exchanging unit HU(1), thereby cooling the substream S_{V2}(1), obtaining a cooled stream S_{CV}(1), wherein more preferably the stream S_{CV}(1) is recycled into R_{U}, more preferably as part of the aqueous liquid stream S_{W} according to (i.1).

Preferably, the process according to said second alternative preferably further comprises recycling at least one of the cooled aqueous streams S_{CV}(j), more preferably all cooled aqueous streams S_{CV}(j) obtained according to (iii) into R_{U}, more preferably as part of the aqueous liquid stream S_{W} according to (i.1).

Said second alternative of the second aspect of the present invention is shown in FIG. 5.

According to a third alternative of the second aspect of the present invention, the process preferably comprises
(ii) subjecting the aqueous liquid stream S_{R} obtained according to (i.5) to depressurization in an evaporation unit EU comprising n evaporation sub-units EU(j), obtaining from the sub-unit EU(j) an aqueous vapor stream S_{V}(j) and an aqueous liquid stream S_{L}(j) comprising ε-caprolactam dissolved in water, wherein S_{V}(j) has a temperature T_{V}(j) and a pressure p_{V}(j) and wherein S_{L}(j) has a temperature T_{L}(j) and a pressure p_{L}(j);
   wherein the aqueous liquid stream S_{R} is passed as feed stream into the evaporation sub-unit EU(1);
   wherein for j<n, the aqueous liquid stream S_{L}(j) obtained from the evaporation sub-unit EU(j) is passed as feed stream into the evaporation sub-unit EU(j+1);
   wherein for j<n, T_{V}(j+1)<T_{V}(j); p_{V}(j+1)<p_{V}(j); T_{L}(j+1)<T_{L}(j); and p_{L}(j+1)<p_{L}(j);
   wherein p_{V}(n) is in the range of from 0.95 to 1.5 bar, more preferably in the range of from 1.0 to 1.4 bar; T_{V}(n) is in the range of from 90 to 110° C., more preferably in the range of from 95 to 100° C.; p_{L}(n) is in the range of from 0.95 to 1.5 bar, more preferably in the range of from 1.0 to 1.4 bar; and T_{L}(n) is in the range of from 90 to 110° C., more preferably in the range of from 95 to 105° C.;
(iii) passing the aqueous vapor streams S_{V}(j) obtained according to (ii) through the heat exchanging units HU(j), thereby heating in HU(j) the stream S_{T}(j) having a temperature T_{ST}(j), obtaining the cooled aqueous stream S_{V}(j), S_{CV}(j), having a temperature T_{CV}(j) with T_{CV}(j)<T_{V}(j) and the heated stream S_{T}(j), S_{HT}(j), having a temperature T_{HST}(j) with T_{HST}(j)>T_{ST}(j);
(iv) separating ε-caprolactam from the aqueous liquid stream S_{L}(n) obtained according to (ii) in at least one heat consuming purification unit PU, wherein the heat consumed in PU is at least partially provided by at least one of the heated streams S_{HT}(j);
(v) recycling at least one aqueous liquid stream S_{RE} obtained from at least one heat consuming purification unit PU according to (iv) into R_{U}, more preferably as part of the aqueous liquid stream S_{W} according to (i.1);
   wherein at least one of the streams S_{L}(j), prior to being passed as feed stream into the evaporation sub-unit EU(j+1) according to (ii), is passed through a heat exchanging unit HU_{RE}, thereby cooling the S_{L}(j), wherein in the heat exchanging unit HU_{RE}, at least one of the streams S_{RE} is heated prior to being recycled into R_{U} according to (v).

According to said third alternative, preferably, the at least one of the streams S_{L}(j) which is passed through the heat exchanging unit HU_{RE} is the stream S_{L}(1).

According to said third alternative, preferably, n=2 and
p_{V}(1) is in the range of from 7.5 to 18 bar, more preferably in the range of from 8 to 16 bar;
T_{V}(1) is in the range of from 160 to 210° C., more preferably in the range of from 170 to 200° C.;
p_{L}(1) is in the range of from 7.5 to 18 bar, more preferably in the range of from 8 to 16 bar; and
T_{L}(1) is in the range of from 160 to 210° C., more preferably in the range of from 170 to 200° C.

Preferably, according to said third alternative, the process further comprises recycling at least one of the cooled aqueous streams S_{CV}(j), more preferably all cooled aqueous streams S_{CV}(j) obtained according to (iii) into R_{U}, more preferably as part of the aqueous liquid stream S_{W} according to (i.1).

Said third alternative of the second aspect of the present invention is shown in FIG. 4.

### Heat-consuming Purification Units PU/PU(i)

In the context of the present invention, preferably, according to (iv), the at least one heat consuming purification unit PU comprises a heat consuming purification unit PU(1), or a heat consuming purification unit PU(2), or a heat consuming purification unit PU(1) and a heat consuming purification unit PU(2), more preferably a heat consuming purification unit PU(1) and a heat consuming purification unit PU(2), wherein
the purification unit PU(1) comprises at least one film evaporator, more preferably at least one falling film evaporator, more preferably two falling film evaporators, more preferably two serially coupled falling film evaporators, wherein in PU(1), water is separated from an aqueous stream comprising ε-caprolactam dissolved in water;
the purification unit PU(2) comprises at least one distillation column, more preferably two or three distillation columns, more preferably two or three serially coupled distillation columns, wherein in PU(2), water is removed from an aqueous stream comprising ε-caprolactam dissolved in water.

Preferably, the process comprises passing at least a part of at least one heated stream S_{HT} as heating medium through a heating jacket of the at least one film evaporator of PU(1).

Preferably, the process comprises passing at least a part of at least one heated stream S_{HT} as heating medium through a heat exchanger of an evaporator of at least one distillation column of PU(2), more preferably through a heat exchanger of each of the two or three distillation columns of PU(2), more preferably through a heat exchanger used for sump evaporation of each of the two or three distillation columns of PU(2).

Preferably, the process comprises passing at least a part of at least one heated stream S_{HT} as heating medium through a heating jacket of the at least one film evaporator of PU(2).

Preferably, according to (iv), separating ε-caprolactam from at least one aqueous liquid stream S_{L}, more preferably S_{L}(n), obtained according to (ii), S_{L} having a water concentration c_{L}(H₂O), in at least one heat consuming purification unit PU according to (iv) comprises
(iv.1) passing said at least one aqueous liquid stream S_{L} through a solid-liquid separation SLU, obtaining an aqueous liquid stream S_{LSLU} comprising ε-caprolactam dissolved in water, S_{LSLU} having a water concentration c_{LSLU}(H₂O), and further obtaining a solid;
(iv.2) passing the at least one aqueous liquid stream S_{L}, preferably the aqueous liquid stream S_{LSLU} obtained according to (iv.1), through the purification unit PU(1), obtaining an aqueous liquid stream SL_{PU1}, S_{LPU1} having a water concentration c_{LPU1}(H₂O), wherein c_{LPU1}(H₂O)<c_{L}(H₂O), more preferably c_{LPU1}(H₂O)<c_{LSLU}(H₂O);
(iv.3) passing the aqueous liquid stream S_{LPU1} obtained according to (iv.2) through a separation unit OSU, obtaining an aqueous liquid stream S_{LOSU}, S_{LOSU} having a water concentration c_{LOSU}(H₂O), wherein, in OSU, oligomers of ε-caprolactam which are comprised in S_{LPU1} are separated from S_{LPU1};
(iv.4) passing the aqueous liquid stream S_{LPU1} obtained according to (iv.2), preferably the aqueous liquid stream c_{LOSU}(H₂O) obtained according to (iv.3), through the purification unit PU(2), obtaining a liquid stream C_{LPU2}(H₂O) having a water concentration C_{LPU2}(H₂O), wherein C_{LPU2}(H₂O)<<c_{LPU1}(H₂O), more preferably C_{LPU2}(H₂O)<<C_{OSU}(H₂O).

### Solid-liquid separation unit

Preferably, the solid-liquid separation SLU according to (iv.1) comprises, more preferably consists of, one or more of a centrifuge, a decanter, a decanter centrifuge, and a filter, more preferably one or more of a decanter and a decanter centrifuge.

Preferably, the separation OSU according to (iv.3) comprises a stirred tank reactor and a film evaporator, more preferably a wipe film evaporator, wherein, more preferably, the stirred tank reactor and a film evaporator are serially coupled.

The present invention is further illustrated by the following set of embodiments and combinations of embodiments resulting from the dependencies and back-references as indicated. In particular, it is noted that in each instance where a range of embodiments is mentioned, for example in the context of a term such as “The process of any one of embodiments 1 to 4”, every embodiment in this range is meant to be explicitly disclosed for the skilled person, i.e. the wording of this term is to be understood by the skilled person as being synonymous to “The process of any one of embodiments 1, 2, 3 and 4”. Further, it is explicitly noted that the following set of embodiments represents a suitably structured part of the general description directed to preferred aspects of the present invention, and, thus, suitably supports, but does not represent the claims of the present invention.

1. A heat-integrated process for hydrolytically depolymerizing a polyamide prepared from ε-caprolactam, said polyamide being contained in a solid material M, the process comprising
(i) preparing an aqueous liquid stream S_{R} comprising ε-caprolactam dissolved in water, comprising
   (i.1) providing the solid material M comprising the polyamide, and providing a aqueous liquid stream S_{W};
   (i.2) preparing a mixture of the solid material M and the aqueous liquid stream S_{W} provided according to (i.1);
   (i.3) preparing an aqueous mixture M_{A} comprising the polyamide dissolved in water from the mixture prepared according to (i.2);
   (i.4) subjecting the aqueous mixture M_{A} prepared according to (i.3) to depolymerization conditions comprising a depolymerization temperature T_{D} at a depolymerization pressure p_{D} in a chemical reactor unit R_{U}, obtaining a liquid aqueous reaction mixture M_{R} in R_{U}, M_{R} comprising ε-caprolactam dissolved in water, wherein T_{D} is in the range of from 230 to 320° C. and p_{D} is in the range of from 40 to 120 bar;
   (i.5) removing an aqueous liquid stream S_{R} from R_{U}, S_{R} comprising ε-caprolactam dissolved in water, wherein said aqueous liquid stream S_{R} has a pressure p_{R} in the range of from 40 to 120 bar and a temperature T_{R} in the range of from 230 to 320° C.;
   (i.6) optionally passing the aqueous liquid stream S_{R} through a heat exchanging unit HU_{O}, obtaining an aqueous liquid stream S_{LO} having a temperature T_{LO} with T_{LO}<T_{R} and having a pressure p_{LO} in the range of from 40 to 120 bar;
(ii) subjecting the aqueous liquid stream S_{R} obtained according to (i.5) or the aqueous liquid stream S_{LO} obtained according to (i.6) to depressurization in an evaporation unit EU, obtaining from EU at least one aqueous vapor stream S_{V} having a temperature T_{V} in the range of from 100 to 230° C., and at least one aqueous liquid stream S_{L} comprising ε-caprolactam dissolved in water;
(iii) passing at least one aqueous vapor stream S_{V} obtained according to (ii), having a temperature T_{V}, through a heat exchanging unit HU, thereby heating in HU at least one stream S_{T} having a temperature T_{ST}, obtaining a cooled aqueous stream S_{V}, S_{CV}, having a temperature T_{CV} with T_{CV}<T_{V} and at least one heated stream S_{T}, S_{HT}, having a temperature T_{HST} with T_{HST}>T_{ST};
(iv) separating ε-caprolactam from at least one aqueous liquid stream S_{L} obtained according to (ii) in at least one heat consuming purification unit PU, wherein the heat consumed in PU is at least partially provided by at least one heated stream S_{HT};
(v) recycling at least one aqueous liquid stream S_{RE} obtained from at least one heat consuming purification unit PU according to (iv) into R_{U}, preferably as part of the aqueous liquid stream S_{W} according to (i.1).

2. The process of embodiment 1, wherein according to (i.5), the aqueous liquid stream S_{R} has a pressure p_{R} in the range of from 50 to 100 bar, preferably in the range of from 60 to 90 bar.

3. The process of embodiment 1 or 2, wherein according to (i.5), the aqueous liquid stream SR has a temperature T_{R} in the range of from 250 to 310° C., more preferably in the range of from 270 to 300° C.

4. The process of any one of embodiments 1 to 3, wherein the solid material M provided according to (i.1) comprises, preferably consists of, waste material, wherein said waste material preferably comprises textile waste material.

5. The process of any one of embodiments 1 to 4, comprising
(ii) subjecting the aqueous liquid stream S_{R} obtained according to (i.5) or the aqueous liquid stream S_{LO} obtained according to (i.6) to depressurization in an evaporation unit EU comprising n evaporation sub-units EU(j), 1≤j≤ n with n≥1, obtaining from each sub-unit EU(j) an aqueous vapor stream S_{V}(j) and an aqueous liquid stream S_{L}(j) comprising ε-caprolactam dissolved in water, wherein S_{V}(j) has a temperature T_{V}(j) and a pressure p_{V}(j) and wherein S_{L}(j) has a temperature T_{L}(j) and a pressure p_{L}(j);
(iii) passing at least one aqueous vapor stream S_{V}(j) obtained according to (ii) and having a temperature T_{V}(j) in the range of from 100 to 230° C., through a heat exchanging unit HU(j), thereby heating in HU(j) a stream S_{T}(j) having a temperature T_{ST}(j), obtaining a cooled aqueous stream S_{V}(j), S_{CV}(j), having a temperature T_{CV}(j) with T_{CV}(j)<T_{V}(j) and at least one heated stream S_{T}(j), S_{HT}(j), having a temperature T_{HST}(j) with T_{HST}(j)>T_{ST}(j);
(iv) separating ε-caprolactam from at least one aqueous liquid stream S_{L}(j) obtained according to (ii) in at least one heat consuming purification unit PU, wherein the heat consumed in PU is at least partially provided by at least one heated stream S_{HT}(j);
(v) recycling at least one aqueous liquid stream S_{RE} obtained from at least one heat consuming purification unit PU according to (iv) into R_{U}, preferably as part of the aqueous liquid stream S_{W} according to (i.1);
wherein at least one evaporation sub-unit EU(j), preferably every evaporation sub-unit EU(j) comprises, more preferably consists of a flash drum.

6. The process of embodiment 5, wherein according to (ii), the aqueous liquid stream SR obtained according to (i.5) or the aqueous liquid stream S_{LO} obtained according to (i.6) is subjected to depressurization in the evaporation unit EU(1); and wherein according to (iv), ε-caprolactam is separated from the aqueous liquid stream S_{L}(n) obtained according to (ii) in at least one heat consuming purification unit PU.

7. The process of embodiment 5 or 6, wherein n>1, and wherein for j<n, the process comprises passing the aqueous liquid stream S_{L}(j) obtained from the evaporation sub-unit EU(j) as feed stream into the evaporation sub-unit EU(j+1).

8. The process of any one of embodiments 5 to 7, wherein n>1, and wherein for j<n, T_{V}(j+1)<T_{V}(j); p_{V}(j+1)<p_{V}(j); T_{L}(j+1)<T_{L}(j); and p_{L}(j+1)<p_{L}(j).

9. The process of any one of embodiments 5 to 8, wherein p_{V}(n) is in the range of from 0.90 to 1.5 bar, preferably in the range of from 0.95 to 1.1 bar; T_{V}(n) is in the range of from 90 to 110° C., preferably in the range of from 95 to 100° C.; p_{L}(n) is in the range of from 0.90 to 1.5 bar, preferably in the range of from 0.95 to 1.1 bar; and T_{L}(n) is in the range of from 90 to 110° C., preferably in the range of from 95 to 105° C.

10. The process of any one of embodiments 5 to 9, wherein n is in the range of from 1 to 3, preferably n=1 or n=2, more preferably n=1.

11. The process of embodiment 10, comprising passing the aqueous liquid stream S_{R} through a heat exchanging unit HU_{O} according to (i.6), obtaining an aqueous liquid stream S_{LO} having a temperature T_{LO} with T_{LO}<T_{R} and having a pressure p_{LO} in the range of from 40 to 120 bar; and subjecting the aqueous liquid stream S_{LO} obtained according to (i.6) to depressurization according to (ii).

12. The process of embodiment 11, wherein T_{LO} is in the range of from 130 to 150° C., preferably in the range of from 120 to 140° C. and a pressure p_{LO} in the range of from 40 to 120 bar, preferably in the range of from 50 to 100 bar, more preferably in the range of from 60 to 90 bar.

13. The process of any one of embodiments 10 to 12, wherein the aqueous vapor stream S_{V}(n) obtained from EU(n) has a pressure p_{V}(n) in the range of from 0.95 to 1.5 bar, preferably in the range of from 1.0 to 1.4 bar, and a temperature T_{V}(n) in the range of from 90 to 110° C., preferably in the range of from 95 to 100° C.; and wherein the aqueous liquid stream S_{L}(n) obtained from EU(n) has a pressure p_{L}(n) in the range of from 0.95 to 1.5 bar, preferably in the range of from 1.0 to 1.4 bar, and a temperature T_{L}(n) in the range of from 90 to 110° C., preferably in the range of from 95 to 100° C.

14. The process of any one of embodiments 10 to 13, wherein according to (iv), ε-caprolactam is separated from the aqueous liquid stream S_{L}(1) obtained according to (ii) in at least one heat consuming purification unit PU, wherein the heat consumed in PU is at least partially provided by the heated stream S_{HT}(1).

15. The process of any one of embodiments 10 to 14, further comprising recycling the cooled aqueous stream S_{CV}(1) into R_{U}, preferably as part of the aqueous liquid stream S_{W} according to (i.1).

16. The process of any one of embodiments 5 to 9, wherein the aqueous liquid stream S_{R} is not passed through a heat exchanging unit HU_{O} according to (i.6), wherein according to (ii), the aqueous liquid stream S_{R} obtained according to (i.5) is subjected to depressurization in an evaporation unit EU.

17. The process of embodiment 16, wherein n is in the range of from 2 to 5, preferably in the range of from 2 to 4, more preferably n=2 or n=3.

18. The process of embodiment 17, comprising
(ii) subjecting the aqueous liquid stream S_{R} obtained according to (i.5) to depressurization in an evaporation unit EU comprising n evaporation sub-units EU(j), obtaining from the sub-unit EU(j) an aqueous vapor stream S_{V}(j) and an aqueous liquid stream S_{L}(j) comprising ε-caprolactam dissolved in water, wherein S_{V}(j) has a temperature T_{V}(j) and a pressure p_{V}(j) and wherein S_{L}(j) has a temperature T_{L}(j) and a pressure p_{L}(j);
   wherein for j<n, the aqueous liquid stream S_{L}(j) obtained from the evaporation sub-unit EU(j) is passed as feed stream into the evaporation sub-unit EU(j+1);
   wherein for j<n, T_{V}(j+1)<T_{V}(j); p_{V}(j+1)<p_{V}(j); T_{L}(j+1)<T_{L}(j); and p_{L}(j+1)<p_{L}(j);
   wherein p_{V}(n) is in the range of from 0.95 to 1.5 bar, preferably in the range of from 1.0 to 1.4 bar; T_{V}(n) is in the range of from 90 to 110° C., preferably in the range of from 95 to 100° C.; p_{L}(n) is in the range of from 0.95 to 1.5 bar, preferably in the range of from 1.0 to 1.4 bar; and T_{L}(n) is in the range of from 90 to 110° C., preferably in the range of from 95 to 105° C.;
(iii) passing at least the aqueous vapor stream S_{V}(n) obtained according to (ii) through the heat exchanging unit HU(n), thereby heating in HU(n) the stream S_{T}(n) having a temperature T_{ST}(n), obtaining the cooled aqueous stream S_{V}(n), S_{CV}(n), having a temperature T_{CV}(n) with T_{CV}(n)<T_{V}(n) and the heated stream S_{T}(n), S_{HT}(n), having a temperature T_{HST}(n) with T_{HST}(n)>T_{ST}(n);
(iv) separating ε-caprolactam from the aqueous liquid stream S_{L}(n) obtained according to (ii) in at least one heat consuming purification unit PU, wherein the heat consumed in PU is at least partially provided by at least one of the heated streams S_{HT}(j);
(v) recycling at least one aqueous liquid stream S_{RE} obtained from at least one heat consuming purification unit PU according to (iv) into R_{U}, preferably as part of the aqueous liquid stream S_{W} according to (i.1).

19. The process of embodiment 18, wherein according to (iii), at least the aqueous vapor streams S_{V}(j) for j=2 . . . n obtained according to (ii) are passed through the heat exchanging units HU(j), j=2 . . . n.

20. The process of any one of embodiments 16 to 19, comprising
(ii) subjecting the aqueous liquid stream S_{R} obtained according to (i.5) to depressurization in an evaporation unit EU comprising n evaporation sub-units EU(j), obtaining from the sub-unit EU(j) an aqueous vapor stream S_{V}(j) and an aqueous liquid stream S_{L}(j) comprising ε-caprolactam dissolved in water, wherein S_{V}(j) has a temperature T_{V}(j) and a pressure p_{V}(j) and wherein S_{L}(j) has a temperature T_{L}(j) and a pressure p_{L}(j);
   wherein the aqueous liquid stream S_{R} is passed as feed stream into the evaporation sub-unit EU(1);
   wherein for j<n, the aqueous liquid stream S_{L}(j) obtained from the evaporation sub-unit EU(j) is passed as feed stream into the evaporation sub-unit EU(j+1);
   wherein for j<n, T_{V}(j+1)<T_{V}(j); p_{V}(j+1)<p_{V}(j); T_{L}(j+1)<T_{L}(j); and p_{L}(j+1)<p_{L}(j);
   wherein p_{V}(n) is in the range of from 0.95 to 1.5 bar, preferably in the range of from 1.0 to 1.4 bar; T_{V}(n) is in the range of from 90 to 110° C., preferably in the range of from 95 to 100° C.; p_{L}(n) is in the range of from 0.95 to 1.5 bar, preferably in the range of from 0.95 to 1.4 bar; and T_{L}(n) is in the range of from 90 to 110° C., preferably in the range of from 95 to 105° C.;
(iii) passing the aqueous vapor streams S_{V}(j) obtained according to (ii) through the heat exchanging units HU(j), thereby heating in HU(j) the stream S_{T}(j) having a temperature T_{ST}(j), obtaining the cooled aqueous stream S_{V}(j), S_{CV}(j), having a temperature T_{CV}(j) with T_{CV}(j)<T_{V}(j) and the heated stream S_{T}(j), S_{HT}(j), having a temperature T_{HST}(j) with T_{HST}(j)>T_{ST}(j);
(iv) separating ε-caprolactam from the aqueous liquid stream S_{L}(n) obtained according to (ii) in at least one heat consuming purification unit PU, wherein the heat consumed in PU is at least partially provided by at least one of the heated streams S_{HT}(j);
(v) recycling at least one aqueous liquid stream S_{RE} obtained from at least one heat consuming purification unit PU according to (iv) into R_{U}, preferably as part of the aqueous liquid stream S_{W} according to (i.1).

21. The process of embodiment 20, wherein n=2 and wherein
p_{V}(1) is in the range of from 7.5 to 18 bar, preferably in the range of from 8 to 16 bar;
T_{V}(1) is in the range of from 160 to 210° C., preferably in the range of from 170 to 200° C.;
p_{L}(1) is in the range of from 7.5 to 18 bar, preferably in the range of from 8 to 16 bar; and
T_{L}(1) is in the range of from 160 to 210° C., preferably in the range of from 170 to 200° C.

22. The process of embodiment 20, wherein n=3 and wherein
p_{V}(1) is in the range of from 19 to 31 bar, preferably in the range of from 20 to 30 bar;
T_{V}(1) is in the range of from 205 to 240° C., preferably in the range of from 210 to 235° C.;
p_{L}(1) is in the range of from 19 to 31 bar, preferably in the range of from 20 to 30 bar; and
T_{L}(1) is in the range of from 205 to 240° C., preferably in the range of from 210 to 135° C.;
p_{V}(2) is in the range of from 7.5 to 18 bar, preferably in the range of from 8 to 16 bar;
T_{V}(2) is in the range of from 160 to 210° C., preferably in the range of from 170 to 200° C.;
p_{L}(2) is in the range of from 7.5 to 18 bar, preferably in the range of from 8 to 16 bar; and
T_{L}(2) is in the range of from 160 to 210° C., preferably in the range of from 170 to 200° C.; or wherein
p_{V}(1) is in the range of from 7.5 to 18 bar, preferably in the range of from 8 to 16 bar;
T_{V}(1) is in the range of from 160 to 210° C., preferably in the range of from 170 to 200° C.;
p_{L}(1) is in the range of from 7.5 to 18 bar, preferably in the range of from 8 to 16 bar; and
T_{L}(1) is in the range of from 160 to 210° C., preferably in the range of from 170 to 200° C.;
p_{V}(2) is in the range of from 1.5 to 7 bar, preferably in the range of from 2 to 7 bar;
T_{V}(2) is in the range of from 115 to 165° C., preferably in the range of from 120 to 165° C.;
p_{L}(2) is in the range of from 1.5 to 7 bar, preferably in the range of from 2 to 7 bar; and T_{L}(2) is in the range of from 115 to 165° C., preferably in the range of from 120 to 165° C.

23. The process of any one of embodiments 20 to 22, further comprising recycling at least one of the cooled aqueous streams S_{CV}(j), preferably all cooled aqueous streams S_{CV}(j) obtained according to (iii) into R_{U}, preferably as part of the aqueous liquid stream S_{W} according to (i.1).

24. The process of any one of embodiments 16 to 19, comprising
(ii) subjecting the aqueous liquid stream S_{R} obtained according to (i.5) to depressurization in an evaporation unit EU comprising n evaporation sub-units EU(j), obtaining from the sub-unit EU(j) an aqueous vapor stream S_{V}(j) and an aqueous liquid stream S_{L}(j) comprising ε-caprolactam dissolved in water, wherein S_{V}(j) has a temperature T_{V}(j) and a pressure p_{V}(j) and wherein S_{L}(j) has a temperature T_{L}(j) and a pressure p_{L}(j);
   wherein the aqueous liquid stream S_{R} is passed as feed stream into the evaporation sub-unit EU(1);
   wherein for j<n, the aqueous liquid stream S_{L}(j) obtained from the evaporation sub-unit EU(j) is passed as feed stream into the evaporation sub-unit EU(j+1);
   wherein for j<n, T_{V}(j+1)<T_{V}(j); p_{V}(j+1)<p_{V}(j); T_{L}(j+1)<T_{L}(j); and p_{L}(j+1)<p_{L}(j);
   wherein p_{V}(n) is in the range of from 0.95 to 1.5 bar, preferably in the range of from 1.0 to 1.4 bar; T_{V}(n) is in the range of from 90 to 110° C., preferably in the range of from 95 to 100° C.; p_{L}(n) is in the range of from 0.95 to 1.5 bar, preferably in the range of from 1.0 to 1.4 bar; and T_{L}(n) is in the range of from 90 to 110° C., preferably in the range of from 95 to 105° C.;
(iii) passing the aqueous vapor streams S_{V}(j), j>1, obtained according to (ii) through the heat exchanging units HU(j), thereby heating in HU(j) the stream S_{T}(j) having a temperature T_{ST}(j), obtaining the cooled aqueous stream S_{V}(j), S_{CV}(j), having a temperature T_{CV}(j) with T_{CV}(j)<T_{V}(j) and the heated stream S_{T}(j), S_{HT}(j), having a temperature T_{HST}(j) with T_{HST}(j)>T_{ST}(j);
(iv) separating ε-caprolactam from the aqueous liquid stream S_{L}(n) obtained according to (ii) in at least one heat consuming purification unit PU, wherein the heat consumed in PU is at least partially provided by at least one of the heated streams S_{HT}(j);
(v) recycling at least one aqueous liquid stream S_{RE} obtained from at least one heat consuming purification unit PU according to (iv) into R_{U}, preferably as part of the aqueous liquid stream S_{W} according to (i.1);
wherein x % of the stream S_{V}(1) obtained according to (ii) are admixed with at least one of the streams S_{RE} according to (v), 0<x≤100;
wherein, if x≠100, the process comprises dividing the stream S_{V}(1) into a substream S_{V1}(1) and a substream S_{V2}(1), S_{V1}(1) constituting x % of the stream S_{V}(1) and S_{V2}(1) constituting (100-x) % of the stream S_{V}(1), and admixing S_{V1}(1) with at least one of the streams S_{RE} according to (v).

25. The process of embodiment 24, wherein for x=100, the substream S_{V2}(1) is passed through the heat exchanging unit HU(1), thereby heating in HU(1) the stream S_{T}(1) having a temperature T_{ST}(1), obtaining the cooled aqueous stream S_{V1}(1), S_{CV}(1), having a temperature T_{CV}(1) with T_{CV}(1)<T_{V1}(1) and the heated stream S_{T}(1), S_{HT}(1), having a temperature T_{HST}(1) with T_{HST}(1)>T_{ST}(1).

26. The process of embodiment 24 or 25, wherein 25≤x≤100, preferably 50≤x≤100, more preferably 75≤x≤100, wherein more preferably, x=100.

27. The process of any one of embodiments 24 to 26, wherein n=3 and wherein
p_{V}(1) is in the range of from 19 to 31 bar, preferably in the range of from 20 to 30 bar;
T_{V}(1) is in the range of from 205 to 240° C., preferably in the range of from 210 to 235° C.;
p_{L}(1) is in the range of from 19 to 31 bar, preferably in the range of from 20 to 30 bar; and
T_{L}(1) is in the range of from 205 to 240° C., preferably in the range of from 210 to 135° C.;
p_{V}(2) is in the range of from 7.5 to 18 bar, preferably in the range of from 8 to 16 bar;
T_{V}(2) is in the range of from 160 to 210° C., preferably in the range of from 170 to 200° C.;
p_{L}(2) is in the range of from 7.5 to 18 bar, preferably in the range of from 8 to 16 bar; and
T_{L}(2) is in the range of from 160 to 210° C., preferably in the range of from 170 to 200° C.; or wherein
p_{V}(1) is in the range of from 7.5 to 18 bar, preferably in the range of from 8 to 16 bar;
T_{V}(1) is in the range of from 160 to 210° C., preferably in the range of from 170 to 200° C.;
p_{L}(1) is in the range of from 7.5 to 18 bar, preferably in the range of from 8 to 16 bar; and
T_{L}(1) is in the range of from 160 to 210° C., preferably in the range of from 170 to 200° C.;
p_{V}(2) is in the range of from 1.5 to 7 bar, preferably in the range of from 2 to 7 bar;
T_{V}(2) is in the range of from 115 to 165° C., preferably in the range of from 120 to 165° C.;
p_{L}(2) is in the range of from 1.5 to 7 bar, preferably in the range of from 2 to 7 bar; and T_{L}(2) is in the range of from 115 to 165° C., preferably in the range of from 120 to 165° C.

28. The process of any one of embodiments 24 to 27, wherein admixing x % of the stream S_{V}(1) obtained according to (ii) with at least one of the streams S_{RE} according to (v) comprises bubbling x % of the stream S_{V}(1) into the at least one aqueous liquid stream S_{RE}.

29. The process of any one of embodiments 24 to 28, further comprising recycling at least one of the cooled aqueous streams S_{CV}(j), preferably all cooled aqueous streams S_{CV}(j) obtained according to (iii) into R_{U}, preferably as part of the aqueous liquid stream S_{W} according to (i.1).

30. The process of embodiment 24 or 25, wherein 1≤x≤20, preferably 2≤x≤15, more preferably 5≤x≤10.

31. The process of embodiment 30, wherein n=2 and wherein
p_{V}(1) is in the range of from 7.5 to 18 bar, preferably in the range of from 8 to 16 bar;
T_{V}(1) is in the range of from 160 to 210° C., preferably in the range of from 170 to 200° C.;
p_{L}(1) is in the range of from 7.5 to 18 bar, preferably in the range of from 8 to 16 bar; and
T_{L}(1) is in the range of from 160 to 210° C., preferably in the range of from 170 to 200° C.

32. The process of embodiment 30 or 31, wherein admixing x % of the stream S_{V}(1) obtained according to (ii) with at least one of the streams S_{RE} according to (v) comprises passing the substream S_{V1}(1) through a heat exchanging unit HU_{RE}, thereby cooling the substream S_{V1}(1), wherein in the heat exchanging unit HU_{RE}, at least one of the streams S_{RE} is heated prior to being recycled into R_{U} according to (v).

33. The process of any one of embodiments 30 to 32, further comprising recycling at least one of the cooled aqueous streams S_{CV}(j), preferably all cooled aqueous streams S_{CV}(j) obtained according to (iii) into R_{U}, preferably as part of the aqueous liquid stream S_{W} according to (i.1).

34. The process of any one of embodiments 16 to 19, comprising
(ii) subjecting the aqueous liquid stream S_{R} obtained according to (i.5) to depressurization in an evaporation unit EU comprising n evaporation sub-units EU(j), obtaining from the sub-unit EU(j) an aqueous vapor stream S_{V}(j) and an aqueous liquid stream S_{L}(j) comprising ε-caprolactam dissolved in water, wherein S_{V}(j) has a temperature T_{V}(j) and a pressure p_{V}(j) and wherein S_{L}(j) has a temperature T_{L}(j) and a pressure p_{L}(j);
   wherein the aqueous liquid stream S_{R} is passed as feed stream into the evaporation sub-unit EU(1);
   wherein for j<n, the aqueous liquid stream S_{L}(j) obtained from the evaporation sub-unit EU(j) is passed as feed stream into the evaporation sub-unit EU(j+1);
   wherein for j<n, T_{V}(j+1)<T_{V}(j); p_{V}(j+1)<p_{V}(j); T_{L}(j+1)<T_{L}(j); and p_{L}(j+1)<p_{L}(j);
   wherein p_{V}(n) is in the range of from 0.95 to 1.5 bar, preferably in the range of from 1.0 to 1.4 bar; T_{V}(n) is in the range of from 90 to 110° C., preferably in the range of from 95 to 100° C.; p_{L}(n) is in the range of from 0.95 to 1.5 bar, preferably in the range of from 1.0 to 1.4 bar; and T_{L}(n) is in the range of from 90 to 110° C., preferably in the range of from 95 to 105° C.;
(iii) passing the aqueous vapor streams S_{V}(j) obtained according to (ii) through the heat exchanging units HU(j), thereby heating in HU(j) the stream S_{T}(j) having a temperature T_{ST}(j), obtaining the cooled aqueous stream S_{V}(j), S_{CV}(j), having a temperature T_{CV}(j) with T_{CV}(j)<T_{V}(j) and the heated stream S_{T}(j), S_{HT}(j), having a temperature T_{HST}(j) with T_{HST}(j)>T_{ST}(j);
(iv) separating ε-caprolactam from the aqueous liquid stream S_{L}(n) obtained according to (ii) in at least one heat consuming purification unit PU, wherein the heat consumed in PU is at least partially provided by at least one of the heated streams S_{HT}(j);
(v) recycling at least one aqueous liquid stream S_{RE} obtained from at least one heat consuming purification unit PU according to (iv) into R_{U}, preferably as part of the aqueous liquid stream S_{W} according to (i.1);
wherein at least one of the streams S_{L}(j), prior to being passed as feed stream into the evaporation sub-unit EU(j+1) according to (ii), is passed through a heat exchanging unit HU_{RE}, thereby cooling the S_{L}(j), wherein in the heat exchanging unit HU_{RE}, at least one of the streams S_{RE} is heated prior to being recycled into R_{U} according to (v).

35. The process of embodiment 34, wherein the at least one of the streams S_{L}(j) which is passed through the heat exchanging unit HU_{RE} is the stream S_{L}(1).

36. The process of embodiment 34 or 35, wherein n=2 and wherein
p_{V}(1) is in the range of from 7.5 to 18 bar, preferably in the range of from 8 to 16 bar;
T_{V}(1) is in the range of from 160 to 210° C., preferably in the range of from 170 to 200° C.;
p_{L}(1) is in the range of from 7.5 to 18 bar, preferably in the range of from 8 to 16 bar; and
T_{L}(1) is in the range of from 160 to 210° C., preferably in the range of from 170 to 200° C. 37. The process of any one of embodiments 34 to 36, further comprising recycling at least one of the cooled aqueous streams S_{CV}(j), preferably all cooled aqueous streams S_{CV}(j) obtained according to (iii) into R_{U}, preferably as part of the aqueous liquid stream S_{W} according to (i.1).

38. The process of any one of embodiments 1 to 37, wherein according to (iv), the at least one heat consuming purification unit PU comprises a heat consuming purification unit PU(1), or a heat consuming purification unit PU(2), or a heat consuming purification unit PU(1) and a heat consuming purification unit PU(2), preferably a heat consuming purification unit PU(1) and a heat consuming purification unit PU(2), wherein
the purification unit PU(1) comprises at least one film evaporator, preferably at least one falling film evaporator, more preferably two falling film evaporators, more preferably two serially coupled falling film evaporators, wherein in PU(1), water is separated from an aqueous stream comprising ε-caprolactam dissolved in water;
the purification unit PU(2) comprises at least one distillation column, preferably two or three distillation columns, more preferably two or three serially coupled distillation columns, wherein in PU(2), water is removed from an aqueous stream comprising ε-caprolactam dissolved in water.

39. The process of embodiment 38, comprising passing at least a part of at least one heated stream S_{HT} as heating medium through a heating jacket of the at least one film evaporator of PU(1).

40. The process of embodiment 38 or 39, comprising passing at least a part of at least one heated stream S_{HT} as heating medium through a heat exchanger of an evaporator of at least one distillation column of PU(2), preferably through a heat exchanger of each of the two or three distillation columns of PU(2), more preferably through a heat exchanger used for sump evaporation of each of the two or three distillation columns of PU(2).

41. The process of any one of embodiments 38 to 40, comprising passing at least a part of at least one heated stream S_{HT} as heating medium through a heating jacket of the at least one film evaporator of PU(2).

42. The process of any one of embodiments 38 to 41, wherein according to (iv), separating ε-caprolactam from at least one aqueous liquid stream S_{L}, preferably S_{L}(n), obtained according to (ii), S_{L} having a water concentration c_{L}(H₂O), in at least one heat consuming purification unit PU according to (iv) comprises
(iv.1) preferably passing said at least one aqueous liquid stream S_{L} through a solid-liquid separation SLU, obtaining an aqueous liquid stream S_{LSLU} comprising ε-caprolactame dissolved in water, S_{LSLU} having a water concentration c_{LSLU}(H₂O), and further obtaining a solid;
(iv.2) passing the at least one aqueous liquid stream S_{L}, preferably the aqueous liquid stream S_{LSLU} obtained according to (iv.1), through the purification unit PU(1), obtaining an aqueous liquid stream SL_{PU1}, S_{LPU1} having a water concentration c_{LPU1}(H₂O), wherein c_{LPU1}(H₂O)<c_{L}(H₂O), preferably c_{LPU1}(H₂O)<c_{LSLU}(H₂O);
(iv.3) preferably passing the aqueous liquid stream S_{LPU1} obtained according to (iv.2) (iv.3) through a separation unit OSU, obtaining an aqueous liquid stream S_{LOSU}, S_{LOSU} having a water concentration c_{LOSU}(H₂O), wherein in OSU, oligomers of ε-caprolactam which are comprised in S_{LPU1} are separated from S_{LPU1};
(iv.4) passing the aqueous liquid stream S_{LPU1} obtained according to (iv.2), preferably the aqueous liquid stream c_{LOSU}(H₂O) obtained according to (iv.3), through the purification unit PU(2), obtaining a liquid stream C_{LPU2}(H₂O) having a water concentration C_{LPU2}(H₂O), wherein C_{LPU2}(H₂O)<<c_{LPU1}(H₂O), preferably C_{LPU2}(H₂O)<<C_{OSU}(H₂O).

43. The process of any one of embodiments 1 to 42, wherein the solid-liquid separation SLU according to (iv.1) comprises, preferably consists of, one or more of a centrifuge, a decanter, a decanter centrifuge, and a filter, preferably one or more of a decanter and a decanter centrifuge.

44. The process of embodiment 42 or 43, wherein the separation OSU according to (iv.3) comprises a stirred tank reactor and a film evaporator, preferably a wipe film evaporator, wherein more preferably, the stirred tank reactor and a film evaporator are serially coupled.

In the context of the present invention, it is noted that the term “polyamide prepared from ε-caprolactam” as used herein refers to “polyamide 6” being characterized by the formula (—NH—(CH₂)₅—CO—)n.

The term “,bar” as used in the context of the present invention refers to “bar(abs)”, i.e. bar (absolute), sometimes also referred to “bara”.

The term “textile material” covers textile raw materials and non-textile raw materials that are processed by various methods into linear, planar and spatial structures. It concerns the linear textile structures produced from them, such as yarns, twisted yarns and ropes, the sheet-like textile structures, such as woven fabrics, knitted fabrics, braids, stitch-bonded fabrics, nonwovens and felts, and the three-dimensional textile structures, i.e. body structures, such as textile hoses, stockings or textile semi-finished products; and it further concerns those finished products which, using the aforementioned products, are brought into a saleable condition by making up, opening up and/or other operations for onward transmission to the processor, the trade or the end consumer.

The term “textile waste material” covers a textile material as defined above, the inherent value of which has been consumed from the perspective of its current holder and, thus, is an end-of-life material for said holder.

In the context of the present invention, a term “X is one or more of A, B and C”, wherein X is a given feature and each of A, B and C stands for specific realization of said feature, is to be understood as disclosing that X is either A, or B, or C, or A and B, or A and C, or B and C, or A and B and C. In this regard, it is noted that the skilled person is capable of transfer to above abstract term to a concrete example, e.g. where X is a chemical element and A, B and C are concrete elements such as Li, Na, and K, or X is a temperature and A, B and C are concrete temperatures such as 10° C., 20° C., and 30° C. In this regard, it is further noted that the skilled person is capable of extending the above term to less specific realizations of said feature, e.g. “X is one or more of A and B” disclosing that X is either A, or B, or A and B, or to more specific realizations of said feature, e.g. “X is one or more of A, B, C and D”, disclosing that X is either A, or B, or C, or D, or A and B, or A and C, or A and D, or B and C, or B and D, or C and D, or A and B and C, or A and B and D, or B and C and D, or A and B and C and D.

### DESCRIPTION OF THE FIGURES

FIG. 1 is a schematic representation of a production unit used for the process according to preferred embodiments of the invention.

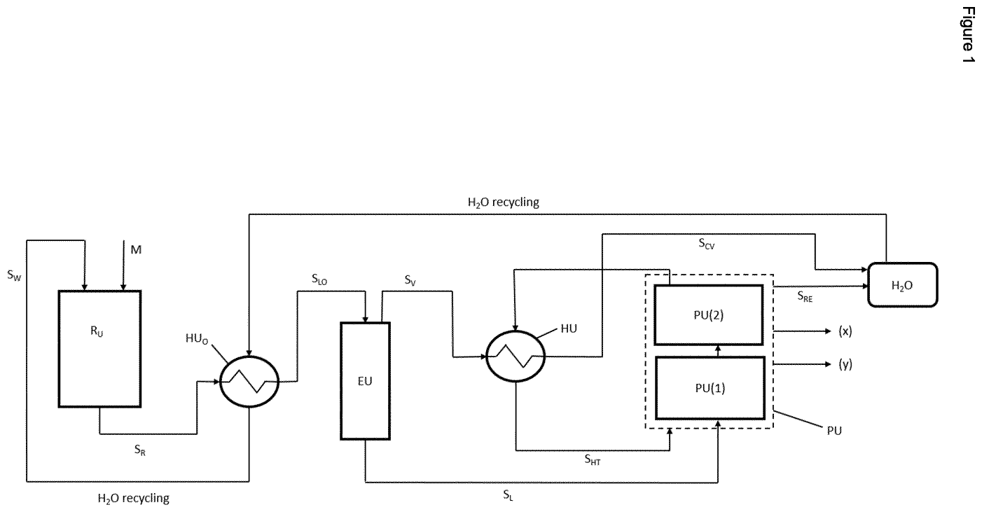

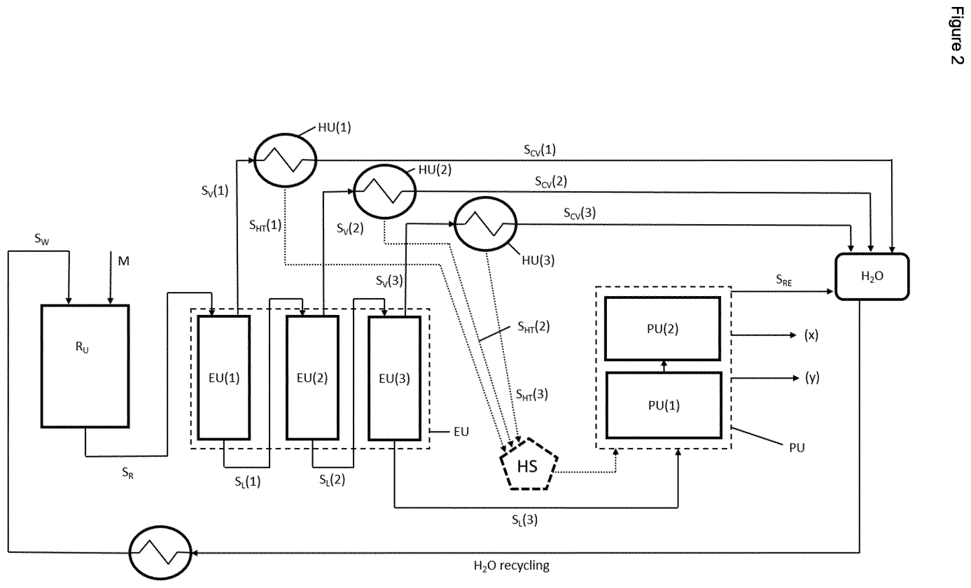

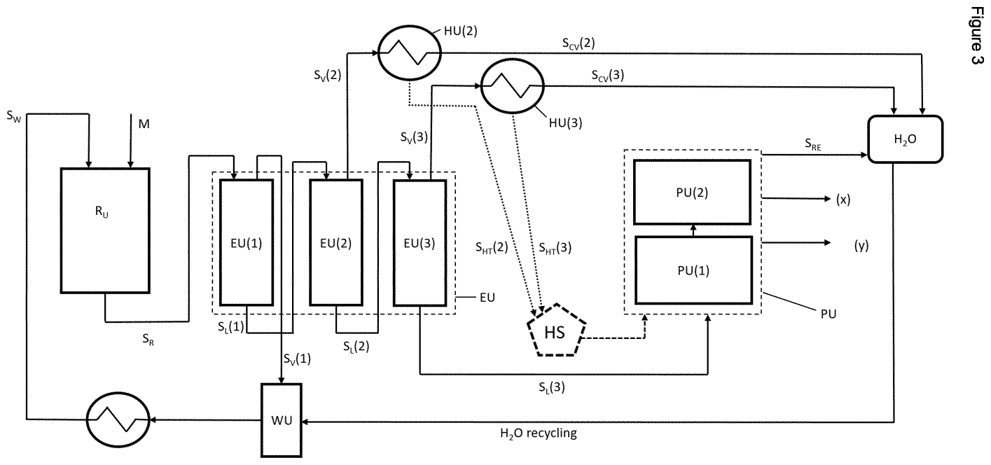

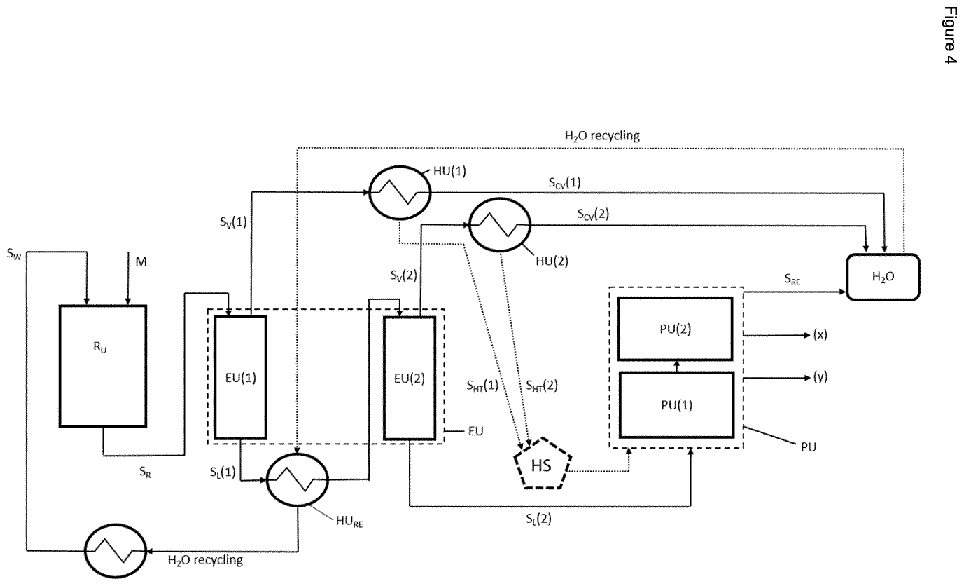

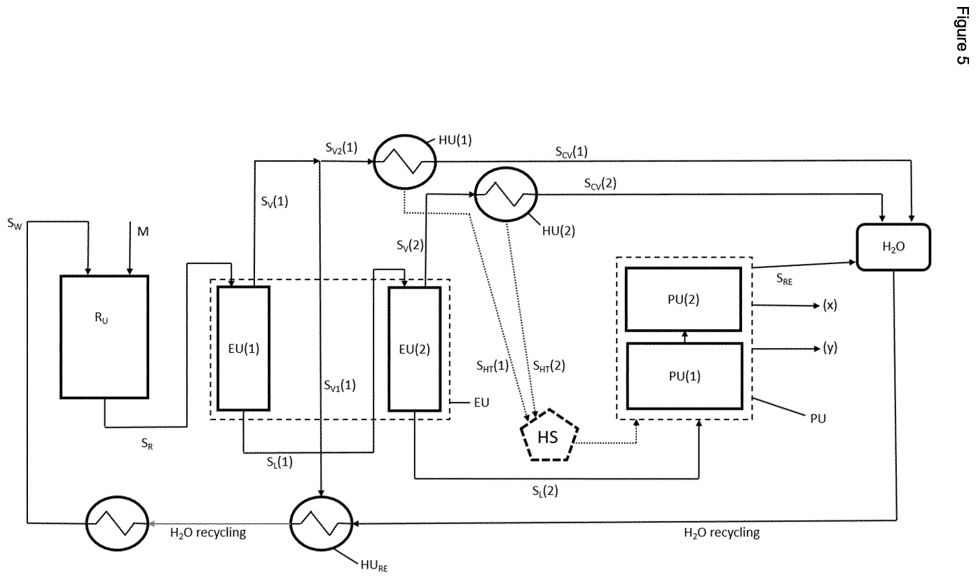

The production unit comprises a reactor unit R_{U}, an evaporation unit EU, a heat consuming purification unit PU and two heat exchanging units HU_{O} and HU. The solid material M comprising the polyamide and an aqueous liquid stream S_{W} are fed into the reactor unit R_{U} and subjected to depolymerization conditions comprising a depolymerization temperature T_{D} at a depolymerization pressure p_{D} as detailed in the foregoing. An aqueous liquid stream S_{R} is removed from the bottom of R_{U}, S_{R} comprising ε-caprolactam dissolved in water, wherein said aqueous liquid stream SR has a pressure p_{R} in the range of from 40 to 120 bar and a temperature T_{R} in the range of from 230 to 320° C. The aqueous liquid stream S_{R} is passed through the heat exchanging unit HU_{O} obtaining an aqueous liquid stream S_{LO} having a temperature T_{LO} with T_{LO}<T_{R} and having a pressure p_{LO} in the range of from 40 to 120 bar. The aqueous liquid stream S_{LO} is subjected to depressurization in the evaporation unit EU. An aqueous vapor stream S_{V} having a temperature T_{V} in the range of from 100 to 230° C. is removed from the top of EU and an aqueous liquid stream S_{L} comprising ε-caprolactam dissolved in water is removed from the bottom of EU. The aqueous vapor stream S_{V}, having a temperature T_{V}, is passed through a heat exchanging unit HU, a cooled aqueous stream S_{CV}, having a temperature T_{CV} with T_{CV}<T_{V}, is removed from HU and a heated stream S_{T}, S_{HT}, having a temperature T_{HST} with T_{HST}>T_{ST}, is also removed from HU. The heated stream S_{T} is used for providing a part of the heat consumed in the heat consuming purification unit PU. The aqueous stream S_{L} comprising ε-caprolactam dissolved in water is fed into PU for separating ε-caprolactam from S_{L}. An aqueous liquid stream S_{RE} (water) is removed from PU and recycled as part of the aqueous liquid stream S_{W}. In particular, water is passed through HU_{O} for heating and then fed to RU as part of the aqueous liquid stream S_{W}. The cooled stream S_{CV}(water) is similarly recycled as part of the aqueous liquid stream S_{W}. PU comprises a heat consuming purification unit PU(1) and a heat consuming purification unit PU(2), wherein the purification unit PU(1) comprises at least one film evaporator and wherein the purification unit PU(2) comprises at least one distillation column. At least one stream (x) comprising ε-caprolactam is removed from PU as well as at least one stream (y) comprising impurities and substantially free of, preferably free of, ε-caprolactam.

FIG. 2 is a schematic representation of a production unit used for the process according to preferred embodiments of the invention.

The production unit comprises a reactor unit R_{U}, an evaporation unit EU comprising three sub-units EU(1), EU(2) and EU(3), a heat consuming purification unit PU and three heat exchanging units HU(1), HU(2) and HU(3). The solid material M comprising the polyamide and an aqueous liquid stream S_{W} are fed into the reactor unit R_{U} and subjected to depolymerization conditions comprising a depolymerization temperature T_{D} at a depolymerization pressure p_{D} as detailed in the foregoing. An aqueous liquid stream S_{R} is removed from the bottom of R_{U}, S_{R} comprising ε-caprolactam dissolved in water, wherein said aqueous liquid stream S_{R} has a pressure p_{R} in the range of from 40 to 120 bar and a temperature T_{R} in the range of from 230 to 320° C. The aqueous liquid stream S_{R} is subjected to depressurization in the evaporation sub-unit EU(1). An aqueous vapor stream S_{V}(1) is removed from the top of EU(1) and an aqueous liquid stream S_{L}(1) comprising ε-caprolactam dissolved in water is removed from the bottom of EU(1), wherein S_{V}(1) has a temperature T_{V}(1) and a pressure p_{V}(1) and wherein S_{L}(1) has a temperature T_{L}(1) and a pressure p_{L}(1). The aqueous vapor stream S_{V}(1), having a temperature T_{V}(1) in the range of from 100 to 230° C., is passed through a heat exchanging unit HU(1), a cooled aqueous stream S_{CV}(1), having a temperature T_{CV}(1) with T_{CV}(1)<T_{V}(1), is removed from HU(1) and a heated stream S_{HT}(1) is also removed from HU(1). The heated stream S_{HT}(1) is used for providing a part of the heat consumed in the heat consuming purification unit PU. The aqueous stream S_{L}(1) comprising ε-caprolactam dissolved in water is fed in the evaporation sub-unit EU(2). An aqueous vapor stream S_{V}(2) is removed from the top of EU(2) and an aqueous liquid stream S_{L}(2) comprising ε-caprolactam dissolved in water is removed from the bottom of EU(2), wherein S_{V}(2) has a temperature T_{V}(2) and a pressure p_{V}(2) and wherein S_{L}(2) has a temperature T_{L}(2) and a pressure p_{L}(2). The aqueous vapor stream S_{V}(2), having a temperature T_{V}(2) in the range of from 100 to 230° C., is passed through a heat exchanging unit HU(2), a cooled aqueous stream S_{CV}(2), having a temperature T_{CV}(2) with T_{CV}(2)<T_{V}(2), is removed from HU(2) and a heated stream S_{HT}(2) is also removed from HU(2). The heated stream S_{HT}(2) is used for providing a part of the heat consumed in the heat consuming purification unit PU. The aqueous stream S_{L}(2) comprising ε-caprolactam dissolved in water is fed in the evaporation sub-unit EU(3). An aqueous vapor stream S_{V}(3) is removed from the top of EU(3) and an aqueous liquid stream S_{L}(3) comprising ε-caprolactam dissolved in water is removed from the bottom of EU(3), wherein S_{V}(3) has a temperature T_{V}(3) and a pressure p_{V}(3) and wherein S_{L}(3) has a temperature T_{L}(3) and a pressure p_{L}(3). The aqueous vapor stream S_{V}(3), having a temperature T_{V}(3) in the range of from 100 to 230° C., is passed through a heat exchanging unit HU(3), a cooled aqueous stream S_{CV}(3), having a temperature T_{CV}(3) with T_{CV}(3)<T_{V}(3), is removed from HU(3) and a heated stream S_{HT}(3) is also removed from HU(3). The heated stream S_{HT}(3) is used for providing a part of the heat consumed in the heat consuming purification unit PU. The aqueous stream S_{L}(3) comprising ε-caprolactam dissolved in water is fed into PU for separating ε-caprolactam from S_{L}(3). An aqueous liquid stream S_{RE} (water) is removed from PU and recycled as part of the aqueous liquid stream S_{W}. The cooled streams S_{CV}(1), S_{CV}(2) and S_{CV}(3) (water) are recycled as part of the aqueous liquid stream S_{W}. Prior to be recycled, these cooled streams can be passed through an optional heat exchanger shown in FIG. 2. PU comprises a heat consuming purification unit PU(1) and a heat consuming purification unit PU(2), wherein the purification unit PU(1) comprises at least one film evaporator and wherein the purification unit PU(2) comprises at least one distillation column. At least one stream (x) comprising ε-caprolactam ε-caprolactam is removed from PU as well as at least one stream (y) comprising impurities and substantially free of, preferably free of, ε-caprolactam.

FIG. 3 is a schematic representation of a production unit used for the process according to preferred embodiments of the invention.

The production unit comprises a reactor unit R_{U}, an evaporation unit EU comprising three sub-units EU(1), EU(2) and EU(3), a heat consuming purification unit PU, two heat exchanging units HU(2) and HU(3) and a water unit WU (a closed loop water system) in which stream S_{V}(1) is bubbled in the liquid water stream). The solid material M comprising the polyamide and an aqueous liquid stream S_{W} are fed into the reactor unit R_{U} and subjected to depolymerization conditions comprising a depolymerization temperature T_{D} at a depolymerization pressure p_{D} as detailed in the foregoing. An aqueous liquid stream S_{R} is removed from the bottom of R_{U}, SR comprising ε-caprolactam dissolved in water, wherein said aqueous liquid stream S_{R} has a pressure p_{R} in the range of from 40 to 120 bar and a temperature T_{R} in the range of from 230 to 320° C. The aqueous liquid stream S_{R} is subjected to depressurization in the evaporation sub-unit EU(1). An aqueous vapor stream S_{V}(1) is removed from the top of EU(1) and an aqueous liquid stream S_{L}(1) comprising ε-caprolactam dissolved in water is removed from the bottom of EU(1), wherein S_{V}(1) has a temperature T_{V}(1) and a pressure p_{V}(1) and wherein S_{L}(1) has a temperature T_{L}(1) and a pressure p_{L}(1). The aqueous vapor stream S_{V}(1), having a temperature T_{V}(1) in the range of from 100 to 230° C., is passed through the water unit WU and water is recycled as part of S_{W}. The aqueous stream S_{L}(1) comprising ε-caprolactam dissolved in water is fed in the evaporation sub-unit EU(2). An aqueous vapor stream S_{V}(2) is removed from the top of EU(2) and an aqueous liquid stream S_{L}(2) comprising ε-caprolactam dissolved in water is removed from the bottom of EU(2), wherein S_{V}(2) has a temperature T_{V}(2) and a pressure p_{V}(2) and wherein S_{L}(2) has a temperature T_{L}(2) and a pressure p_{L}(2). The aqueous vapor stream S_{V}(2), having a temperature T_{V}(2) in the range of from 100 to 230° C., is passed through a heat exchanging unit HU(2), a cooled aqueous stream S_{CV}(2), having a temperature T_{CV}(2) with T_{CV}(2)<T_{V}(2), is removed from HU(2) and a heated stream S_{HT}(2) is also removed from HU(2). The heated stream S_{HT}(2) is used for providing a part of the heat consumed in the heat consuming purification unit PU. The aqueous stream S_{L}(2) comprising ε-caprolactam dissolved in water is fed in the evaporation sub-unit EU(3). An aqueous vapor stream S_{V}(3) is removed from the top of EU(3) and an aqueous liquid stream S_{L}(3) comprising ε-caprolactam dissolved in water is removed from the bottom of EU(3), wherein S_{V}(3) has a temperature T_{V}(3) and a pressure p_{V}(3) and wherein S_{L}(3) has a temperature T_{L}(3) and a pressure p_{L}(3). The aqueous vapor stream S_{V}(3), having a temperature T_{V}(3) in the range of from 100 to 230° C., is passed through a heat exchanging unit HU(3), a cooled aqueous stream S_{CV}(3), having a temperature T_{CV}(3) with T_{CV}(3)<T_{V}(3), is removed from HU(3) and a heated stream S_{HT}(3) is also removed from HU(3). The heated stream S_{HT}(3) is used for providing a part of the heat consumed in the heat consuming purification unit PU. The aqueous stream S_{L}(3) comprising ε-caprolactam dissolved in water is fed into PU for separating ε-caprolactam from S_{L}(3). An aqueous liquid stream S_{RE} (water) is removed from PU and recycled as part of the aqueous liquid stream S_{W} after having been passed through WU. The cooled streams S_{CV}(2) and S_{CV}(3) (water) are recycled as part of the aqueous liquid stream S_{W} by passing through WU, and optionally through a heat exchanger downstream of WU (shown in FIG. 3). PU comprises a heat consuming purification unit PU(1) and a heat consuming purification unit PU(2), wherein the purification unit PU(1) comprises at least one film evaporator and wherein the purification unit PU(2) comprises at least one distillation column. At least one stream (x) comprising ε-caprolactam is removed from PU as well as at least one stream (y) comprising impurities and substantially free of, preferably free of, ε-caprolactam.

FIG. 4 is a schematic representation of a production unit used for the process according to preferred embodiments of the invention.

The production unit comprises a reactor unit R_{U}, an evaporation unit EU comprising two sub-units EU(1) and EU(2), a heat consuming purification unit PU and three heat exchanging units HU(1), HU(2) and HU_{RE}. The solid material M comprising the polyamide and an aqueous liquid stream S_{W} are fed into the reactor unit R_{U} and subjected to depolymerization conditions comprising a depolymerization temperature T_{D} at a depolymerization pressure p_{D} as detailed in the foregoing. An aqueous liquid stream S_{R} is removed from the bottom of R_{U}, S_{R} comprising ε-caprolactam dissolved in water, wherein said aqueous liquid stream S_{R} has a pressure p_{R} in the range of from 40 to 120 bar and a temperature T_{R} in the range of from 230 to 320° C. The aqueous liquid stream S_{R} is subjected to depressurization in the evaporation sub-unit EU(1). An aqueous vapor stream S_{V}(1) is removed from the top of EU(1) and an aqueous liquid stream S_{L}(1) comprising ε-caprolactam dissolved in water is removed from the bottom of EU(1), wherein S_{V}(1) has a temperature T_{V}(1) and a pressure p_{V}(1) and wherein S_{L}(1) has a temperature T_{L}(1) and a pressure p_{L}(1). The aqueous vapor stream S_{V}(1), having a temperature T_{V}(1) in the range of from 100 to 230° C., is passed through a heat exchanging unit HU(1), a cooled aqueous stream S_{CV}(1), having a temperature T_{CV}(1) with T_{CV}(1)<T_{V}(1), is removed from HU(1) and a heated stream S_{HT}(1) is also removed from HU(1). The heated stream S_{HT}(1) is used for providing a part of the heat consumed in the heat consuming purification unit PU. The aqueous stream S_{L}(1) comprising ε-caprolactam dissolved in water is passed through a heat exchanging unit HU_{RE} prior to be fed in the evaporation sub-unit EU(2). An aqueous vapor stream S_{V}(2) is removed from the top of EU(2) and an aqueous liquid stream S_{L}(2) comprising ε-caprolactam dissolved in water is removed from the bottom of EU(2), wherein S_{V}(2) has a temperature T_{V}(2) and a pressure p_{V}(2) and wherein S_{L}(2) has a temperature T_{L}(2) and a pressure p_{L}(2). The aqueous vapor stream S_{V}(2), having a temperature T_{V}(2) in the range of from 100 to 230° C., is passed through a heat exchanging unit HU(2), a cooled aqueous stream S_{CV}(2), having a temperature T_{CV}(2) with T_{CV}(2)<T_{V}(2), is removed from HU(2) and a heated stream S_{HT}(2) is also removed from HU(2). The heated stream S_{HT}(2) is used for providing a part of the heat consumed in the heat consuming purification unit PU. The aqueous stream S_{L}(2) comprising ε-caprolactam dissolved in water is fed into PU for separating ε-caprolactam from S_{L}(2). An aqueous liquid stream S_{RE} (water) is removed from PU and recycled as part of the aqueous liquid stream S_{W} by passing through HU_{RE}. The cooled streams S_{CV}(1) and S_{CV}(2) (water) are recycled as part of the aqueous liquid stream S_{W} by passing through HU_{RE}, and optionally through a heat exchanger downstream of HU_{RE} (shown in FIG. 4). PU comprises a heat consuming purification unit PU(1) and a heat consuming purification unit PU(2), wherein the purification unit PU(1) comprises at least one film evaporator and wherein the purification unit PU(2) comprises at least one distillation column. At least one stream (x) comprising ε-caprolactam is removed from PU as well as at least one stream (y) comprising impurities and substantially free of, preferably free of, ε-caprolactam.

FIG. 5 is a schematic representation of a production unit used for the process according to preferred embodiments of the invention.

The production unit comprises a reactor unit R_{U}, an evaporation unit EU comprising two sub-units EU(1) and EU(2), a heat consuming purification unit PU and three heat exchanging units HU(1), HU(2) and HU_{RE}. The solid material M comprising the polyamide and an aqueous liquid stream S_{W} are fed into the reactor unit R_{U} and subjected to depolymerization conditions comprising a depolymerization temperature T_{D} at a depolymerization pressure p_{D} as detailed in the foregoing. An aqueous liquid stream S_{R} is removed from the bottom of R_{U}, S_{R} comprising ε-caprolactam dissolved in water, wherein said aqueous liquid stream S_{R} has a pressure p_{R} in the range of from 40 to 120 bar and a temperature T_{R} in the range of from 230 to 320° C. The aqueous liquid stream S_{R} is subjected to depressurization in the evaporation sub-unit EU(1). An aqueous vapor stream S_{V}(1) is removed from the top of EU(1) and an aqueous liquid stream S_{L}(1) comprising ε-caprolactam dissolved in water is removed from the bottom of EU(1), wherein S_{V}(1) has a temperature T_{V}(1) and a pressure p_{V}(1) and wherein S_{L}(1) has a temperature T_{L}(1) and a pressure p_{L}(1). The aqueous vapor stream S_{V}(1) is divided in two sub-streams S_{V1}(1) and S_{V2}(1). S_{V1}(1) constituting x % of the stream S_{V}(1) and S_{V2}(1) constituting (100-x) % of the stream S_{V}(1), with x≠100. S_{V1}(1) is passed through a heat exchanging unit HU_{RE} and admixed with S_{RE} to be recycled as part of the aqueous liquid stream S_{W}. S_{V2}(1) is passed through a heat exchanging unit HU(1), a cooled aqueous stream S_{CV}(1), having a temperature T_{CV}(1) with T_{CV}(1)<T_{V}(1), is removed from HU(1) and a heated stream S_{HT}(1) is also removed from HU(1). The heated stream S_{HT}(1) is used for providing a part of the heat consumed in the heat consuming purification unit PU. The aqueous stream S_{L}(1) comprising ε-caprolactam dissolved in water is fed in the evaporation sub-unit EU(2). An aqueous vapor stream S_{V}(2) is removed from the top of EU(2) and an aqueous liquid stream S_{L}(2) comprising ε-caprolactam dissolved in water is removed from the bottom of EU(2), wherein S_{V}(2) has a temperature T_{V}(2) and a pressure p_{V}(2) and wherein S_{L}(2) has a temperature T_{L}(2) and a pressure p_{L}(2). The aqueous vapor stream S_{V}(2), having a temperature T_{V}(2) in the range of from 100 to 230° C., is passed through a heat exchanging unit HU(2), a cooled aqueous stream S_{CV}(2), having a temperature T_{CV}(2) with T_{CV}(2)<T_{V}(2), is removed from HU(2) and a heated stream S_{HT}(2) is also removed from HU(2). The heated stream S_{HT}(2) is used for providing a part of the heat consumed in the heat consuming purification unit PU. The aqueous stream S_{L}(2) comprising ε-caprolactam dissolved in water is fed into PU for separating ε-caprolactam from S_{L}(2). An aqueous liquid stream S_{RE} (water) is removed from PU and recycled as part of the aqueous liquid stream S_{W} by passing through HU_{RE}. The cooled streams S_{CV}(1) and S_{CV}(2) (water) are recycled as part of the aqueous liquid stream S_{W} by passing through HU_{RE}, and optionally through a heat exchanger downstream of HU_{RE} (shown in FIG. 5). PU comprises a heat consuming purification unit PU(1) and a heat consuming purification unit PU(2), wherein the purification unit PU(1) comprises at least one film evaporator and wherein the purification unit PU(2) comprises at least one distillation column. At least one stream (x) comprising ε-caprolactam is removed from PU as well as at least one stream (y) comprising impurities and substantially free of, preferably free of, ε-caprolactam.

1.-18. (canceled)

19. A heat-integrated process for hydrolytically depolymerizing a polyamide prepared from ε-caprolactam, said polyamide being contained in a solid material M, the process comprising
(i) preparing an aqueous liquid stream S_{R} comprising ε-caprolactam dissolved in water, comprising
(i.1) providing the solid material M comprising the polyamide, and providing a aqueous liquid stream S_{W};
(i.2) preparing a mixture of the solid material M and the aqueous liquid stream S_{W} provided according to (i.1);
(i.3) preparing an aqueous mixture M_{A} comprising the polyamide dissolved in water from the mixture prepared according to (i.2);
(i.4) subjecting the aqueous mixture M_{A} prepared according to (i.3) to depolymerization conditions comprising a depolymerization temperature T_{D} at a depolymerization pressure p_{D} in a chemical reactor unit R_{U}, obtaining a liquid aqueous reaction mixture M_{R} in R_{U}, M_{R} comprising ε-caprolactam dissolved in water, wherein T_{D} is in the range of from 230 to 320° C. and p_{D} is in the range of from 40 to 120 bar;
(i.5) removing an aqueous liquid stream S_{R} from R_{U}, S_{R} comprising ε-caprolactam dissolved in water, wherein said aqueous liquid stream S_{R} has a pressure p_{R} in the range of from 40 to 120 bar and a temperature T_{R} in the range of from 230 to 320° C.;
(i.6) optionally passing the aqueous liquid stream S_{R} through a heat exchanging unit HU_{O}, obtaining an aqueous liquid stream S_{LO} having a temperature T_{LO} with T_{LO}<T_{R} and having a pressure pro in the range of from 40 to 120 bar;
(ii) subjecting the aqueous liquid stream S_{R} obtained according to (i.5) or the aqueous liquid stream S_{LO} obtained according to (i.6) to depressurization in an evaporation unit EU, obtaining from EU at least one aqueous vapor stream S_{V} having a temperature T_{V} in the range of from 100 to 230° C., and at least one aqueous liquid stream S_{L} comprising ε-caprolactam dissolved in water;
(iii) passing at least one aqueous vapor stream S_{V} obtained according to (ii), having a temperature T_{V}, through a heat exchanging unit HU, thereby heating in HU at least one stream S_{T} having a temperature T_{ST}, obtaining a cooled aqueous stream S_{V}, S_{CV}, having a temperature T_{CV} with T_{CV}<T_{V} and at least one heated stream S_{T}, S_{HT}, having a temperature T_{HST} with T_{HST}>T_{ST};
(iv) separating ε-caprolactam from at least one aqueous liquid stream S_{L} obtained according to (ii) in at least one heat consuming purification unit PU, wherein the heat consumed in PU is at least partially provided by at least one heated stream S_{HT};
(v) recycling at least one aqueous liquid stream S_{RE} obtained from at least one heat consuming purification unit PU according to (iv) into R_{U}, preferably as part of the aqueous liquid stream S_{W} according to (i.1).

20. The process of claim 19, wherein according to (i.5), the aqueous liquid stream S_{R} has a pressure p_{R} in the range of from 50 to 100 bar.

21. The process of claim 19, comprising
(ii) subjecting the aqueous liquid stream S_{R} obtained according to (i.5), or the aqueous liquid stream S_{LO} obtained according to (i.6), to depressurization in an evaporation unit EU comprising n evaporation sub-units EU(j), 1≤j≤n with n≥1, obtaining from each sub-unit EU(j) an aqueous vapor stream S_{V}(j) and an aqueous liquid stream S_{L}(j) comprising ε-caprolactam dissolved in water, wherein S_{V}(j) has a temperature T_{V}(j) and a pressure p_{V}(j) and wherein S_{L}(j) has a temperature T_{L}(j) and a pressure p_{L}(j);
(iii) passing at least one aqueous vapor stream S_{V}(j) obtained according to (ii) and having a temperature T_{V}(j) in the range of from 100 to 230° C., through a heat exchanging unit HU(j), thereby heating in HU(j) a stream S_{T}(j) having a temperature T_{ST}(j), obtaining a cooled aqueous stream S_{V}(j), S_{CV}(j), having a temperature T_{CV}(j) with T_{CV}(j)<T_{V}(j) and at least one heated stream S_{T}(j), S_{HT}(j), having a temperature T_{HST}(j) with T_{HST}(j)>T_{ST}(j);
(iv) separating ε-caprolactam from at least one aqueous liquid stream S_{L}(j) obtained according to (ii) in at least one heat consuming purification unit PU, wherein the heat consumed in PU is at least partially provided by at least one heated stream S_{HT}(j);
(v) recycling at least one aqueous liquid stream S_{RE} obtained from at least one heat consuming purification unit PU according to (iv) into R_{U};
wherein at least one evaporation sub-unit EU(j) comprises a flash drum.

22. The process of claim 21, wherein according to (ii), the aqueous liquid stream S_{R} obtained according to (i.5), or the aqueous liquid stream S_{LO} obtained according to (i.6), is subjected to depressurization in the evaporation unit EU(1); and wherein according to (iv), ε-caprolactam is separated from the aqueous liquid stream S_{L}(n) obtained according to (ii) in at least one heat consuming purification unit PU.

23. The process of claim 21, wherein n>1, and wherein for j<n, the process comprises passing the aqueous liquid stream S_{L}(j) obtained from the evaporation sub-unit EU(j) as feed stream into the evaporation sub-unit EU(j+1).

24. The process of claim 19, wherein n is in the range of from 1 to 3;
wherein the process comprises passing the aqueous liquid stream S_{R} through a heat exchanging unit HU_{O} according to (i.6), obtaining an aqueous liquid stream S_{LO} having a temperature T_{LO} with T_{LO}<T_{R} and having a pressure pro in the range of from 40 to 120 bar; and subjecting the aqueous liquid stream S_{LO} obtained according to (i.6) to depressurization according to (ii).

25. The process of claim 19, wherein the aqueous liquid stream S_{R} is not passed through a heat exchanging unit HU_{O} according to (i.6), wherein according to (ii), the aqueous liquid stream S_{R} obtained according to (i.5) is subjected to depressurization in an evaporation unit EU.

26. The process of claim 25, wherein n is in the range of from 2 to 5; and
wherein the process comprises
(ii) subjecting the aqueous liquid stream S_{R} obtained according to (i.5) to depressurization in an evaporation unit EU comprising n evaporation sub-units EU(j), obtaining from the sub-unit EU(j) an aqueous vapor stream S_{V}(j) and an aqueous liquid stream S_{L}(j) comprising ε-caprolactam dissolved in water, wherein S_{V}(j) has a temperature T_{V}(j) and a pressure p_{V}(j) and wherein S_{L}(j) has a temperature T_{L}(j) and a pressure p_{L}(j);
wherein for j<n, the aqueous liquid stream S_{L}(j) obtained from the evaporation sub-unit EU(j) is passed as feed stream into the evaporation sub-unit EU(j+1);
wherein for j<n, T_{V}(j+1)<T_{V}(j); p_{V}(j+1)<p_{V}(j); T_{L}(j+1)<T_{L}(j); and p_{L}(j+1)<p_{L}(j);
wherein p_{V}(n) is in the range of from 0.95 to 1.5 bar; T_{V}(n) is in the range of from 90 to 110° C.; p_{L}(n) is in the range of from 0.95 to 1.5 bar; and T_{L}(n) is in the range of from 90 to 110° C.;
(iii) passing at least the aqueous vapor stream S_{V}(n) obtained according to (ii) through the heat exchanging unit HU(n), thereby heating in HU(n) the stream S_{T}(n) having a temperature T_{ST}(n), obtaining the cooled aqueous stream S_{V}(n), S_{CV}(n), having a temperature T_{CV}(n) with T_{CV}(n)<T_{V}(n) and the heated stream S_{T}(n), S_{HT}(n), having a temperature T_{HST}(n) with T_{HST}(n)>T_{ST}(n);
(iv) separating ε-caprolactam from the aqueous liquid stream S_{L}(n) obtained according to (ii) in at least one heat consuming purification unit PU, wherein the heat consumed in PU is at least partially provided by at least one of the heated streams S_{HT}(j);
(v) recycling at least one aqueous liquid stream S_{RE} obtained from at least one heat consuming purification unit PU according to (iv) into R_{U}.

27. The process of claim 25, comprising
(ii) subjecting the aqueous liquid stream S_{R} obtained according to (i.5) to depressurization in an evaporation unit EU comprising n evaporation sub-units EU(j), obtaining from the sub-unit EU(j) an aqueous vapor stream S_{V}(j) and an aqueous liquid stream S_{L}(j) comprising ε-caprolactam dissolved in water, wherein S_{V}(j) has a temperature T_{V}(j) and a pressure p_{V}(j) and wherein S_{L}(j) has a temperature T_{L}(j) and a pressure p_{L}(j);
wherein the aqueous liquid stream S_{R} is passed as feed stream into the evaporation sub-unit EU(1);
wherein for j<n, the aqueous liquid stream S_{L}(j) obtained from the evaporation sub-unit EU(j) is passed as feed stream into the evaporation sub-unit EU(j+1);
wherein for j<n, T_{V}(j+1)>T_{V}(j); p_{V}(j+1)<p_{V}(j); T_{L}(j+1)<T_{L}(j); and p_{L}(j+1)<p_{L}(j);
wherein p_{V}(n) is in the range of from 0.95 to 1.5 bar; T_{V}(n) is in the range of from 90 to 110° C.; p_{L}(n) is in the range of from 0.95 to 1.5 bar; and T_{L}(n) is in the range of from 90 to 110° C.;
(iii) passing the aqueous vapor streams S_{V}(j) obtained according to (ii) through the heat exchanging units HU(j), thereby heating in HU(j) the stream S_{T}(j) having a temperature T_{ST}(j), obtaining the cooled aqueous stream S_{V}(j), S_{CV}(j), having a temperature T_{CV}(j) with T_{CV}(j)<T_{V}(j) and the heated stream S_{T}(j), S_{HT}(j), having a temperature T_{HST}(j) with T_{HST}(j)>T_{ST}(j);
(iv) separating ε-caprolactam from the aqueous liquid stream S_{L}(n) obtained according to (ii) in at least one heat consuming purification unit PU, wherein the heat consumed in PU is at least partially provided by at least one of the heated streams S_{HT}(j);
(v) recycling at least one aqueous liquid stream S_{RE} obtained from at least one heat consuming purification unit PU according to (iv) into R_{U}.

28. The process of claim 27, wherein n=2 and wherein
p_{V}(1) is in the range of from 7.5 to 18 bar;
T_{V}(1) is in the range of from 160 to 210° C.;
p_{L}(1) is in the range of from 7.5 to 18 bar;
or
wherein n=3 and wherein
p_{V}(1) is in the range of from 19 to 31 bar;
T_{V}(1) is in the range of from 205 to 240° C.;
p_{L}(1) is in the range of from 19 to 31 bar; and T_{L}(1) is in the range of from 205 to 240° C.;
p_{V}(2) is in the range of from 7.5 to 18 bar;
T_{V}(2) is in the range of from 160 to 210° C.;
p_{L}(2) is in the range of from 7.5 to 18 bar; and T_{L}(2) is in the range of from 160 to 210° C.;
or
wherein n=3 and wherein
p_{V}(1) is in the range of from 7.5 to 18 bar;
T_{V}(1) is in the range of from 160 to 210° C.;
p_{L}(1) is in the range of from 7.5 to 18 bar; and T_{L}(1) is in the range of from 160 to 210° C.;
p_{V}(2) is in the range of from 1.5 to 7 bar;
T_{V}(2) is in the range of from 115 to 165° C.;
p_{L}(2) is in the range of from 1.5 to 7 bar; and T_{L}(2) is in the range of from 115 to 165° C.

29. The process of claim 25, comprising
(ii) subjecting the aqueous liquid stream S_{R} obtained according to (i.5) to depressurization in an evaporation unit EU comprising n evaporation sub-units EU(j), obtaining from the sub-unit EU(j) an aqueous vapor stream S_{V}(j) and an aqueous liquid stream S_{L}(j) comprising ε-caprolactam dissolved in water, wherein S_{V}(j) has a temperature T_{V}(j) and a pressure p_{V}(j) and wherein S_{L}(j) has a temperature T_{L}(j) and a pressure p_{L}(j);
wherein the aqueous liquid stream S_{R} is passed as feed stream into the evaporation sub-unit EU(1);
wherein for j<n, the aqueous liquid stream S_{L}(j) obtained from the evaporation sub-unit EU(j) is passed as feed stream into the evaporation sub-unit EU(j+1);
wherein for j<n, T_{V}(j+1)<T_{V}(j); p_{V}(j+1)<p_{V}(j); T_{L}(j+1)<T_{L}(j); and p_{L}(j+1)<p_{L}(j);
wherein p_{V}(n) is in the range of from 0.95 to 1.5 bar; T_{V}(n) is in the range of from 90 to 110° C.; p_{L}(n) is in the range of from 0.95 to 1.5 bar; and T_{L}(n) is in the range of from 90 to 110° C.;
(iii) passing the aqueous vapor streams S_{V}(j), j>1, obtained according to (ii) through the heat exchanging units HU(j), thereby heating in HU(j) the stream S_{T}(j) having a temperature T_{ST}(j), obtaining the cooled aqueous stream S_{V}(j), S_{CV}(j), having a temperature T_{CV}(j) with T_{CV}(j)<T_{V}(j) and the heated stream S_{T}(j), S_{HT}(j), having a temperature T_{HST}(j) with T_{HST}(j)>T_{ST}(j);
(iv) separating ε-caprolactam from the aqueous liquid stream S_{L}(n) obtained according to (ii) in at least one heat consuming purification unit PU, wherein the heat consumed in PU is at least partially provided by at least one of the heated streams S_{HT}(j);
(v) recycling at least one aqueous liquid stream S_{RE} obtained from at least one heat consuming purification unit PU according to (iv) into R_{U}, preferably as part of the aqueous liquid stream S_{W} according to (i.1);
wherein x % of the stream S_{V}(1) obtained according to (ii) are admixed with at least one of the streams S_{RE} according to (v), 0<x≤100;
wherein, if x≠100, the process comprises dividing the stream S_{V}(1) into a substream S_{V1}(1) and a substream S_{V2}(1), S_{V1}(1) constituting x % of the stream S_{V}(1) and S_{V2}(1) constituting (100-x) % of the stream S_{V}(1), and admixing S_{V1}(1) with at least one of the streams S_{RE} according to (v).

30. The process of claim 29, wherein 25≤x≤100.

31. The process of claim 29, wherein n=3 and wherein
p_{V}(1) is in the range of from 19 to 31 bar;
T_{V}(1) is in the range of from 205 to 240° C.;
p_{L}(1) is in the range of from 19 to 31 bar; and T_{L}(1) is in the range of from 205 to 240° C.;
p_{V}(2) is in the range of from 7.5 to 18 bar;
T_{V}(2) is in the range of from 160 to 210° C.;
p_{L}(2) is in the range of from 7.5 to 18 bar; and T_{L}(2) is in the range of from 160 to 210° C.;
or wherein
p_{V}(1) is in the range of from 7.5 to 18 bar;
T_{V}(1) is in the range of from 160 to 210° C.;
p_{L}(1) is in the range of from 7.5 to 18 bar; and T_{L}(1) is in the range of from 160 to 210° C.;
p_{V}(2) is in the range of from 1.5 to 7 bar;
T_{V}(2) is in the range of from 115 to 165° C.;
p_{L}(2) is in the range of from 1.5 to 7 bar; and T_{L}(2) is in the range of from 115 to 165° C.

32. The process of claim 29, wherein 1≤x≤20.

33. The process of claim 25, comprising
(ii) subjecting the aqueous liquid stream S_{R} obtained according to (i.5) to depressurization in an evaporation unit EU comprising n evaporation sub-units EU(j), obtaining from the sub-unit EU(j) an aqueous vapor stream S_{V}(j) and an aqueous liquid stream S_{L}(j) comprising ε-caprolactam dissolved in water, wherein S_{V}(j) has a temperature T_{V}(j) and a pressure p_{V}(j) and wherein S_{L}(j) has a temperature T_{L}(j) and a pressure p_{L}(j);
wherein the aqueous liquid stream S_{R} is passed as feed stream into the evaporation sub-unit EU(1);
wherein for j<n, the aqueous liquid stream S_{L}(j) obtained from the evaporation sub-unit EU(j) is passed as feed stream into the evaporation sub-unit EU(j+1);
wherein for j<n, T_{V}(j+1)<T_{V}(j); p_{V}(j+1)<p_{V}(j); T_{L}(j+1)<T_{L}(j); and p_{L}(j+1)<p_{L}(j);
wherein p_{V}(n) is in the range of from 0.95 to 1.5 bar; T_{V}(n) is in the range of from 90 to 110° C.; p_{L}(n) is in the range of from 0.95 to 1.5 bar; and T_{L}(n) is in the range of from 90 to 110° C.;
(iii) passing the aqueous vapor streams S_{V}(j) obtained according to (ii) through the heat exchanging units HU(j), thereby heating in HU(j) the stream S_{T}(j) having a temperature T_{ST}(j), obtaining the cooled aqueous stream S_{V}(j), S_{CV}(j), having a temperature T_{CV}(j) with T_{CV}(j)<T_{V}(j) and the heated stream S_{T}(j), S_{HT}(j), having a temperature T_{HST}(j) with T_{HST}(j)>T_{ST}(j);
(iv) separating ε-caprolactam from the aqueous liquid stream S_{L}(n) obtained according to (ii) in at least one heat consuming purification unit PU, wherein the heat consumed in PU is at least partially provided by at least one of the heated streams S_{HT}(j);
(v) recycling at least one aqueous liquid stream S_{RE} obtained from at least one heat consuming purification unit PU according to (iv) into R_{U};
wherein at least one of the streams S_{L}(j), prior to being passed as feed stream into the evaporation sub-unit EU(j+1) according to (ii), is passed through a heat exchanging unit HU_{RE}, thereby cooling the stream S_{L}(j), wherein in the heat exchanging unit HU_{RE}, at least one of the streams S_{RE} is heated prior to being recycled into R_{U} according to (v).

34. The process of claim 33, wherein the at least one of the streams S_{L}(j) which is passed through the heat exchanging unit HU_{RE} is the stream S_{L}(1).

35. The process of claim 19, wherein according to (iv), the at least one heat consuming purification unit PU comprises a heat consuming purification unit PU(1), or a heat consuming purification unit PU(2), or a heat consuming purification unit PU(1) and a heat consuming purification unit PU(2), wherein
the purification unit PU(1) comprises at least one film evaporator, wherein in PU(1), water is separated from an aqueous stream comprising ε-caprolactam dissolved in water;
the purification unit PU(2) comprises at least one distillation column, wherein in PU(2), water is removed from an aqueous stream comprising ε-caprolactam dissolved in water.

36. The process of claim 35, wherein according to (iv), separating ε-caprolactam from at least one aqueous liquid stream S_{L}, obtained according to (ii), S_{L} having a water concentration c_{L}(H₂O), in at least one heat consuming purification unit PU according to (iv) comprises
(iv.1) optionally passing said at least one aqueous liquid stream S_{L} through a solid-liquid separation SLU, obtaining an aqueous liquid stream S_{LSLU} comprising ε-caprolactam dissolved in water, S_{LSLU} having a water concentration c_{LSLU}(H₂O), and further obtaining a solid;
(iv.2) passing the at least one aqueous liquid stream S_{L} obtained according to (iv.1), through the purification unit PU(1), obtaining an aqueous liquid stream SL_{PU1}, S_{LPU1} having a water concentration c_{LPU1}(H₂O), wherein c_{LPU1}(H₂O)<c_{L}(H₂O), preferably c_{LPU1}(H₂O)<c_{LSLU}(H₂O);
(iv.3) optionally passing the aqueous liquid stream S_{LPU1} obtained according to (iv.2) through a separation unit OSU, obtaining an aqueous liquid stream S_{LOSU}, S_{LOSU} having a water concentration c_{LOSU}(H₂O), wherein in OSU, oligomers of ε-caprolactam which are comprised in S_{LPU1} are separated from S_{LPU1};
(iv.4) passing the aqueous liquid stream S_{LPU1} obtained according to (iv.2), or the aqueous liquid stream c_{LOSU}(H₂O) obtained according to (iv.3), through the purification unit PU(2), obtaining a liquid stream C_{LPU2}(H₂O) having a water concentration C_{LPU2}(H₂O), wherein C_{LPU2}(H₂O)<<C_{LPU1}(H₂O), preferably C_{LPU2}(H₂O)<<c_{OSU}(H₂O).
